(19) United States
(12) Patent Application Publication
(10) Pub. No.: US 2008/0095849 A1
(43) Pub. Date: Apr. 24, 2008

(54) Peptide sustained release compositions and uses thereof

(75) Inventors: Cindy W. Wu, Canoga Park, CA (US); Paul Burke, Oxnard, CA (US); Merrill S. Goldenberg, Thousand Oaks, CA (US); Daxian Shan, Oxnard, CA (US)

Correspondence Address: FOLEY & LARDNER LLP, 150 EAST GILMAN STREET, P.O. BOX 1497, MADISON, WI, 53701-1497, US

(73) Assignee: Amgen Inc.

(21) Appl. No.: 11/411,464

(22) Filed: Apr. 25, 2006

Related U.S. Application Data
(60) Provisional application No. 60/674,872, filed on Apr. 25, 2005.

(51) Int. Cl. A61K 9/14; A61K 38/08; A61P 29/00

(52) U.S. Cl. 424/486; 514/15

(57) ABSTRACT

Sustained delivery compositions that modulate the release of incorporated prophylactic, therapeutic, and/or diagnostic agents, and methods of preparation and use thereof, are disclosed. In particular embodiments, the compositions include a polymeric matrix; a prophylactic, therapeutic, and/or diagnostic agent dispersed and/or dissolved within the polymeric matrix; and a carbohydrate component that is separately dispersed within the polymeric matrix. The carbohydrate component modulates the release of the incorporated agent from the polymeric matrix. The compositions can be prepared by dissolving a biocompatible polymer in a solvent to form a polymer solution, and separately dispersing a carbohydrate and a prophylactic, therapeutic, and/or diagnostic agent within the polymer solution. The polymer solution is then solidified to form a polymeric matrix, wherein a significant amount of the carbohydrates is dispersed in the polymeric matrix separately from the incorporated agent. In particular embodiments, the compositions include a polymeric matrix and a B1 peptide antagonist dispersed within the polymeric matrix.

This application claims the benefit of U.S. Provisional Application No. 60/674,872 filed Apr. 25, 2005, which is incorporated by reference herein.

### FIELD OF THE INVENTION

The present invention relates broadly to the field of sustained delivery formulations. More specifically, the invention describes sustained delivery formulations of proteins or peptides. Additionally, the invention includes compositions and methods relating to formulating and using prophylactic and therapeutic peptides in polymeric microparticles containing separately dispersed carbohydrate porogens. In one embodiment, the invention provides a sustained delivery composition comprising a poly(lactide-co-glycolide) copolymer matrix having a B1 peptide antagonist dissolved and/or dispersed therein and a carbohydrate porogen separately dispersed therein.

### BACKGROUND OF THE INVENTION

In recent years, increasingly sophisticated and potent protein-based and peptide-based drugs have been developed by the biotech industry. However, the prophylactic and/or therapeautic use of many other protein- or peptide-based compounds, has been hampered because of their susceptibility to proteolytic breakdown, rapid plasma clearance, peculiar dose-response curves, immunogenicity, bioincompatibility, and/or the tendency of peptides and proteins to undergo aggregation, adsorption, and/or denaturation. These characteristics often render traditional methods of drug delivery ineffective or sub-optimal when applied to protein or peptide based drugs. Therefore, an immense amount of interest has been increasingly placed on controlled and/or sustained release drug delivery systems to maintain the therapeutic efficacy or diagnostic value of these important classes of biologically active agents.

One of the primary goals of sustained delivery systems is to maintain the levels of an active agent within an effective range and ideally at a constant level. One approach for sustained delivery of an active agent is by microencapsulation, in which the active agent is enclosed within a polymeric matrix. The importance of biocompatible and/or biodegradable polymers as carriers for parenteral drug delivery systems is now well established. Biocompatible, biodegradable, and relatively inert substances such as poly(lactide) (PLA) or poly(lactide-co-glycolide) (PLG) structures such as microparticles or films containing the active agent to be administered are commonly employed sustained delivery devices (for review, see M. Chasin, Biodegradable polymers for controlled drug delivery. In: J. 0. Hollinger Editor, Biomedical Applications of Synthetic Biodegradable Polymers CRC, Boca Raton, Fla. (1995), pp. 1-15; T. Hayashi, Biodegradable polymers for biomedical uses. Prog. Polym. Sci. 194 (1994), pp. 663-700; and Harjit Tamber, P\dot{a}l Johansen, Hans P. Merkle and Bruno Gander, Formulation aspects of biodegradable polymeric microspheres for antigen delivery Advanced Drug Delivery Reviews, Volume 57, Issue 3, 10 Jan. 2005, Pages 357-376). A relatively steady release of one or more active agents incorporated within such polymers is possible because of the degradation profile of these polymers in an aqueous environment. By encapsulating active agents in a polymer matrix in various forms such as microparticles and/or films the active agent is released at a relatively slow rate over a prolonged time. Achieving sustained drug release in such a manner may afford less frequent administration, thereby increasing patient compliance and reducing discomfort; protection of the therapeutic compound within the body; potentially optimized prophylactic or therapeutic responses and prolonged efficacy; and avoidance of peak-related side-effects by maintaining more-constant blood levels of the active agent. Furthermore, these compositions can oftentimes be administered by injection, allowing for localized delivery and high local concentrations of the active agents.

Unfortunately, there still exist many challenges to the design of polymer based sustained release delivery systems for protein- and peptide-based therapeutics. A basic requirement for such delivery systems is that the materials used are acceptable for parenteral application. Another critical requirement is sufficiently good control of the release of the encapsulated active agent. It is generally important to maintain the concentration of the active agent within an effective window for a time period sufficient to achieve the desired effect and to avoid excessive concentrations, which may lead to side effects or untoward results. It is often difficult to achieve the desired release kinetics with monolithic microparticles as the fraction of the active agent released within the first day after administration is often dependent on the loading level of the drug.

Another fundamental requirement for developing an effective sustained polymer based sustained delivery device for the delivery of macromolecules, is that the integrity of the active agent must be adequately maintained during manufacture. This is often a difficult challenge as most protein and peptide drugs are dependent on a three dimensional conformation for their bioactivity and that conformation can easily be compromised. For example, most of the polymers that are used to manufacture controlled release parenteral preparations are not soluble in water and consequently the protein or peptide is exposed to an organic solvent in the encapsulation step.

Examples of other undesirable stresses that are associated with manufacturing of controlled release preparations that may compromise the integrity of any particular protein or peptide include high shear forces used to form droplets of the polymer solution in an continuous phase, exposure to polymerization reactions, high temperatures, and undesirably low or high pH values.

Similarly, another requirement is that the integrity of the active agent, particularly proteins or peptides, is retained within the microparticles during release. Depending on the chosen duration of release, this period can be anywhere from a few days up to several months. Although the prior art describes various sustained delivery compositions and methods for making them (for example, U.S. Pat. Nos. 5,916,597 and 6,748,866 both issued to Tracy, et al.; U.S. Pat. No. 5,019,400, issued to Gombotz, et al.; U.S. Pat. No. 5,922,253, issued to Herbert, et al.; and U.S. Pat. No. 6,531,154, issued to Mathiowitz, et al.), the in vivo release of incorporated active agents from biocompatible, biodegradable polymers is, in many cases, non-uniform throughout the life of the delivery device and tend to provide long term sustained delivery ranging from a few weeks to many months.

Therefore, there continues to exist a need for the development of new and improved polymer based sustained delivery compositions that rely on the use of commercially available and widely accepted as being safe biocompatible and/or biodegradable polymers, allowing for shorter term release profiles with low levels of burst release, and addressing the various other drug delivery challenges posed by active agents, such as proteins and peptides.

### SUMMARY OF THE INVENTION

This invention relates to sustained release compositions that provide for the relative uniform release of biologically active agents incorporated therein in a defined pattern over a desireable period when the composition is parenterally administered to a mammal.

One exemplary aspect of the present invention includes sustained delivery compositions that provide for the accelerated sustained release of one or more proteins or peptides incorporated therein in a defined pattern over a period of time of about three days to about three weeks when the compositions are parenterally administered to a mammal. Such compositions may include a biocompatible and/or biodegradable polymeric matrix, a prophylactic, therapeutic, and/or diagnostic protein or peptide dissolved and/or dispersed within the polymeric matrix, and a carbohydrate component that is dispersed within the polymeric matrix. The carbohydrate component modulates the release of the incorporated active agent from the polymeric matrix in a relatively accelerated manner over a period of time up to about three weeks.

The invention features pharmaceutical compositions comprising active agents, particularly peptides (but not limited to peptides) in an formulation for relatively shortened extended release, one which is capable of releasing the active agent, e.g., peptide, over a predetermined release period of between about 3 days and about 21 days in an effective amount.

Another exemplary aspect of the invention relates to methods for the preparation of particular accelerated sustained delivery compositions comprising the steps of dissolving a biocompatible and/or biodegradable polymer in a solvent to form a polymer solution, dispersing and/or dissolving at least one protein or peptide therein, dispersing a carbohydrate within the polymer/protein or polymer/peptide mixture, causing the solution to form a polymeric matrix wherein the carbohydrate component is dispersed in the polymeric matrix separately from the incorporated active agent, and extracting residual solvents from the composition. The carbohydrate component modulates the release of the incorporated active agent from the polymeric matrix in a relatively consistent manner over a period of time of between about three days and about three weeks when therein in a defined pattern over a period three days to about three weeks when the composition is parenterally administered to a mammal.

According to yet another aspect of the invention, a kit comprising a pharmaceutical composition herein is provided. In certain embodiments, the kit includes a container containing a single dose of a pharmaceutical composition comprising microparticles containing an active agent for treating a condition that is treatable by the accelerated sustained delivery of the active agent from the microparticles. The number of microparticles provided by the single dose will be dependent upon the amount of active agent present in each microparticle and the period of time over which sustained delivery is desired. Preferably, the single dose is selected to achieve the accelerated sustained delivery of the active agent over a period of about three days to about 21 days, wherein the single dose of microparticles is selected to achieve the desired release profile for treating the condition.

According to another aspect of the invention, a syringe containing any of the sustained delivery compositions disclosed herein is provided. For example, the syringe may contain a single dose of the sustained delivery composition, preferably microparticles, containing an active agent for treating a condition that is treatable by the sustained delivery of the active agent from the sustained delivery composition. In certain embodiments of the invention, a needle is attached to the syringe, wherein the needle has a bore size that is from 14 to 30 gauge.

Another aspect of the present invention relates to methods of using the novel compositions of the present invention in the prevention or treatment of a disease, condition, or disorder.

These and other aspects of the invention will be described in greater detail below. Throughout this disclosure, all technical and scientific terms have the same meaning as commonly understood by one of ordinary skill in the art to which this invention pertains unless defined otherwise.

### BRIEF DESCRIPTION OF THE DRAWINGS

FIG. 1 shows that inclusion of a carbohydrate (formulated with salt) porogen accelerates in vivo release rate of Peptide A from microparticles.

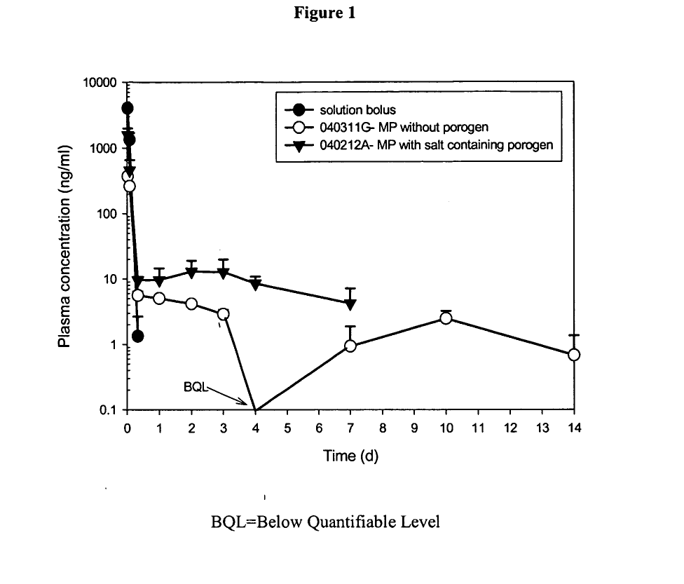

FIG. 2 depicts plasma concentration as a function of time and illustrates that a salt-free carbohydrate porogen also accelerates in vivo release rate of Peptide A from microparticles as compared to microparticle with salt containing porogen.

FIG. 3 shows measurable Peptide A plasma concentration levels in rats for ˜10 days for PLGA/salt free porogen-encapsulated Peptide A microparticle as compared to ˜14 days for PLGA encapsulated Peptide A microparticles.

FIG. 4 shows measurable Peptide A plasma concentration levels in rats for ˜10 days for PLGA/salt free porogen-encapsulated Peptide A microparticles as compared to ˜14 days for PLGA encapsulated Peptide A microparticles.

FIG. 5 shows measurable Peptide B plasma concentration levels in rats for 10-14 days for PLGA/porogen-encapsulated Peptide B microparticle (Lot #43815-030320H). As a comparison, plasma concentration-time profiles are plotted for the solution bolus of Peptide B and PLGA-encapsulated Peptide B microparticle (Lot #43815-030506A), which show release profiles for 8 hours and a month, respectively.

FIG. 6 shows measurable Peptide A plasma concentration levels in rats for ˜10 days for PLGA/Methylcellulose porogen-encapsulated Peptide A microparticle as previously observed with PLGA/Trehalose porogen-based MP.

FIG. 7 shows comparable pharmacokinetic profiles with measurable Peptide A plasma concentration levels in rats for ˜10 days for PLGA/salt free porogen microparticles fabricated by both the spray-dray and spray-freeze dry processes.

FIG. 8 shows that microparticles fabricated with low methanol ratio results in a reduction in the in vivo burst (as defined by maximum plasma concentration, Cmax), as well as, an increase in sustained plasma level of Peptide A.

FIG. 9 shows the cumulative fraction release of Peptide A at t=24 hr (IVR burst) as a function of porogen load for 10% drug load and 15% drug load formulation; illustrating an increase in burst with porogen and drug load increases.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar to those described herein can be used in the practice or testing of the present invention, suitable methods and materials are described below. In addition, the materials, methods, and examples are illustrative only and not intended to be limiting.

Each of the patents, applications and articles cited herein, and each document cited or referenced therein, including during the prosecution of any of the patents and/or applications cited herein (“patent cited documents”), and any manufacturer's instructions or catalogues for any products cited herein or mentioned in any of the references and in any of the patent cited documents, are hereby incorporated herein by reference. Documents incorporated by reference into this text or any teachings therein may be used in the practice of this invention. Documents incorporated by reference into this text are not admitted to be prior art.

As used herein, the words “may” and “may be” are to be interpreted in an open-ended, non-restrictive manner. At minimum, “may” and “may be” are to be interpreted as definitively including structure or acts recited.

Natural amino acid residues are discussed in three ways: full name of the amino acid, standard three-letter code, or standard single-letter code in accordance with the chart shown below.

A = Ala
C = Cys
D = Asp
E = Glu
F = Phe
G = Gly
H = His
I = Ile
K = Lys
L = Leu
M = Met
N = Asn
P = Pro
Q = Gln
R = Arg
S = Ser
T = Thr
V = Val
W = Trp
Y = Tyr

Unless clearly indicated otherwise, use of the term “amino acid” is intended to encompass both natural and unnatural amino acids, as well as both the D- and L-isomer of the amino acid. Abbreviations used herein for unnatural amino acids are the same as described in U.S. Pat. No. 5,834,431, PCT publication WO 98/07746, Neugebauer, W., et al., Kinin B. receptor antagonists with multi-enzymatic resistance properties. Can. J. Physiol. Pharmacol., 80:287-292 (2002), Stewart, et al., Correlation of Secondary Structures of Bradykinin B1 Receptor Antagonists with their Activity. J. of Biolmol. Structure & Dynamics, 4:585-593 (2002), John M. Stewart, Bradykinin antagonists: discovery and development (Review). Peptides, 25:527-532 (2004). For example, the abbreviation “Orn” and “DOrn” is intended to refer to the L- and D-isomer of the unnatural amino acid ornithine; Hyp is trans-4-hydroxy-proline; “Tic” and DTic (or Dtic) is the L- and D- isomer of 1,2,3,4-tetrahydroisoquinoline-3-carboxylic acid, and Cpg is α-cyclopentylglycine. The abbreviation “Dab” and “D-Dab” is intended to refer to the L- and D-isomer of the unnatural amino acid, D-2-aminobutyric acid, respectively. The abbreviation “3′Pal” and “D-3′Pal” is intended to refer to the L- and D-isomer of the unnatural amino acid 3′-pyridylalanine, respectively. Also, the abbreviation “Ig1” is intended to include both “Ig1a” and “Ig1b” (α-(1-indanyl)glycine and α-(2-indanyl)glycine, respectively). Similarly, “DIg1” is intended to include both “D-Ig1a” and “D-Ig1b” (the D-isomers of α-(1-indanyl)glycine and α-(2-indanyl)glycine, respectively). Preferably, when used herein, Ig1 is Ig1b and D-Ig1 is D-Ig1b. The term “B1” means the bradykinin B1 receptor (see, Judith M Hall, A review of BK receptors. Pharmac. Ther. 56:131-190 (1992)). Unless specifically noted otherwise, B1 or bradykinin B1 receptor is intended to mean the human bradykinin B1 receptor (hB1). Preferably, hB1 is the wild-type receptor. More preferably, hB1 is the bradykinin receptor described in GenBank Accession no. AJ238044.

As used herein, the terms “effective amount” when used with reference to a sustained delivery composition of an active agent, e.g., a B1 peptide antagonist, refers to an amount or dosage sufficient to produce a desired result (e.g., for prophylaxis, therapy, or diagnosis with the compositions of the present invention). In the case of sustained delivery compositions comprising B1 peptide antagonists, the desired result may be a desired reduction in inflammation and/or pain, for example, or to support an observable decrease in the level of one or more biological activities mediated by B1. More specifically, a “therapeutically effective amount” of an active agent, e.g., a B1 peptide antagonist, is an amount of that particular agent which is sufficient to inhibit, or halt altogether, for some desired period of time, one or more clinically defined pathological processes associated with the condition at issue, e.g., in the case of B1 peptide antagonists, inflammation and/or pain, in a subject treated in vivo with the agent(s). The effective amount may vary depending on the specific active agent selected, and a variety of other factors and conditions related to the subject to be treated and the severity of the disorder. For example, if the sustained delivery composition comprises one or more peptides such as a B1 peptide antagonist or an analogue, derivative, conjugates and/or complex thereof intended for release upon parenteral administration to a patient, factors such as the age, weight and health of the patient as well as dose response curves and toxicity data obtained in preclinical animal work would be among those considered. If the agent(s) is to be contacted with the cells in vitro, one would also design a variety of pre-clinical in vitro studies to assess such parameters as uptake, half-life, dose, toxicity, etc. The determination of an effective amount or a therapeutically effective amount for a given agent is well within the ability of those skilled in the art.

“Patient” as that term is used herein, refers to the recipient of the treatment. In a specific embodiment, the patient is a mammal, such as a human, canine, murine, feline, bovine, ovine, swine or caprine. In a preferred embodiment, the patient is a human.

The term “pharmaceutically effective” means that a substance so described is determined to have activity that affects a medical parameter or disease state (for example, pain). In the context of the invention, this term may refer to a B1-induced or B1-mediated disease or abnormal medical condition or disorder, and more specifically, to antagonism of inflammation or pain.

The terms “antagonist”, “inhibitor”, and “inverse agonist” (e.g., see, Rianne A. F. de Ligt, et. al, British Journal of Pharmacology, 2000, 130, 131) refer to a molecule that blocks, impedes, reduces, lessens or in some way interferes with the biological activity of the associated protein of interest. An “antagonist” or “inhibitor” as used herein may include a molecule that when formulated and administered as described herein prevents, ameliorates or abolishes inflammation and/or pain as measured in at least one generally accepted in vivo animal model of pain and/or inhibits biochemical challenges in in vivo animal models of edema, inflammation, or pain.

Additionally, further formulations of the compositions of the present invention with physiologically acceptable salts and/or excipients are also encompassed herein. The phrases “physiologically acceptable salts” and “pharmacologically acceptable salts” as used herein are interchangeable are intended to include any salts that are known or later discovered to be pharmaceutically acceptable (i.e., useful in the treatment of a warm-blooded animal). Some specific examples are: acetate; trifluoroacetate; hydrohalides, such as hydrochloride and hydrobromide; sulfate; citrate; tartrate; glycolate; oxalate; salts of inorganic and organic acids, including, but not limited to, hydrochloric acid, hydrobromic acid, sulfuric acid, phosphoric acid, methanesulphonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compositions comprise an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al., J. Pharm. Sci. 66:1 (1977).

The sustained delivery compositions, particularly, microparticles, of the present invention are particularly useful for slow release of active agents with short biological half-lives, such as certain macromolecules such as proteins and peptides. As a result, the sustained delivery compositions described herein may also enable the use of alternative routes of administration when the sustained delivery compositions include a therapeutic drug and are administered to a patient for slow release or targeted delivery of the drug to the site requiring therapy. The slow release of such therapeutic agents is particularly useful for therapeutic proteins or peptides having short half-lives that must be administered by injection. The microparticles are useful for therapy or prophylaxis when the active agent is a therapeutic agent or a pharmaceutical compound that is delivered to a patient and slowly released from the microparticles over time. If the pharmaceutical compound cannot be formed into a particle, then it is complexed to a carrier, such as albumin, and the carrier-pharmaceutical compound complex is formed into a microparticle. The microparticle can either provide for the slow release of the agent throughout the body or the microparticle can include an affinity molecule specific for a target tissue, or tumor, and be injected into a patient for targeted slow release of the therapeutic agent, such as an antitumor, antiviral, antibacterial, antiparasitic, or antiarthritic agent, cytokine, hormone, or insulin directly to the site requiring therapy. As discussed above, the affinity molecule may be cleavable.

As mentioned, the compositions disclosed herein enable prophylactic, therapeutic, and/or diagnostic use of certain classes of active agents some of which were previously considered too unstable in vivo to be used effectively. For example, the known shortcomings in known B1 peptide antagonists with respect to their therapeutic use are surmountable by formulating them in compositions of the present invention that maximize antagonist activity and specificity while prolonging efficacious half-life in vivo. More specifically, the half-life of Peptide A (SEQ ID NO: 15), Peptide B (SEQ ID NO:37) and Peptide C (SEQ ID NO:36) is about 3 hours, 40 minutes, and 40 minutes, respectively, in rat plasma. The accelerated sustained release and/or extended circulating half-lives of the B1 peptides formulated as described herein results in a much more desirable exposure window and may provide better efficacy in vivo as compared to common formulations of these compounds.

The present invention also provides methods of using the accelerated sustained release compositions to deliver B1 peptide antagonists in order to prevent or treat inflammation and/or pain (including, but not limited to, inflammatory pain and associated hyperalgesia and allodynia). Therefore, the compositions of the present invention as described herein provides a means for eliciting a prophylactic and/or therapeutic effect in a patient in need thereof by administering a composition comprising poly(lactide-co-glycolide) copolymer and a B1 peptide antagonist, for example. The B1 peptide antagonist compositions of the invention may additionally have therapeutic value for the prevention or treatment of other painful conditions associated with or mediated by B1 activation, including, but not limited to, thalamic pain syndrome, diabetes, toxins and chemotherapy, septic shock, arthritis, mixed-vascular and non-vascular syndromes, general inflammation, arthritis, rheumatic diseases, lupus, osteoarthritis, inflammatory bowel disorders, inflammatory eye disorders, inflammatory or unstable bladder disorders, psoriasis, skin complaints with inflammatory components, sunburn, carditis, inflammatory bowel disease, dermatitis, myositis, neuritis, collagen vascular diseases, chronic inflammatory conditions, epithelial tissue damage or dysfunction, herpes simplex, diabetic neuropathy pain, post-herpetic neuralgia, causalgia, sympathetically maintained pain, deafferentation syndromes, tension headache, angina, migraine, surgical pain, disturbances of visceral motility at respiratory, genitourinary, gastrointestinal or vascular regions, wounds, burns, allergic rhinitis, asthma, allergic skin reactions, pruritis, vitiligo, general gastrointestinal disorders, colitis, gastric ulceration, duodenal ulcers, or vasomotor or allergic rhinitis.

The invention also provides for the use of the compositions of the present invention comprising B1 peptide antagonists for the prevention or treatment of acute pain, dental pain, back pain, lower back pain, pain from trauma, surgical pain, pain resulting from amputation or abscess, causalgia, demyelinating diseases, trigeminal neuralgia, cancer, chronic alcoholism, stroke, thalamic pain syndrome, diabetes, acquired immune deficiency syndrome (“AIDS”), toxins and chemotherapy, general headache, migraine, cluster headache, mixed-vascular and non-vascular syndromes, tension headache, general inflammation, arthritis, rheumatic diseases, lupus, osteoarthritis, inflammatory bowel disorders, inflammatory eye disorders, inflammatory or unstable bladder disorders, psoriasis, skin complaints with inflammatory components, sunburn, carditis, dermatitis, myositis, neuritis, collagen vascular diseases, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, allergic rhinitis, epithelial tissue damage or dysfunction, herpes simplex, post-herpetic neuralgia, disturbances of visceral motility at respiratory, genitourinary, gastrointestinal or vascular regions, wounds, burns, allergic skin reactions, pruritis, vitiligo, general gastrointestinal disorders, colitis, gastric ulceration, duodenal ulcers, and bronchial disorders.

Accordingly, the present invention also relates to the use of one or more of the compositions comprising a B1 peptide antagonist as at least one active agent in the manufacture of a medicament for the treatment of a B1 mediated disorders, diseases and conditions mentioned hereinabove or hereinbelow such as acute pain, dental pain, back pain, lower back pain, pain from trauma, surgical pain, pain resulting from amputation or abscess, causalgia, demyelinating diseases, trigeminal neuralgia, cancer, chronic alcoholism, stroke, thalamic pain syndrome, diabetes, acquired immune deficiency syndrome (“AIDS”), toxins and chemotherapy, general headache, migraine, cluster headache, mixed-vascular and non-vascular syndromes, tension headache, general inflammation, arthritis, rheumatic diseases, lupus, osteoarthritis, inflammatory bowel disorders, inflammatory eye disorders, inflammatory or unstable bladder disorders, psoriasis, skin complaints with inflammatory components, sunburn, carditis, dermatitis, myositis, neuritis, collagen vascular diseases, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, allergic rhinitis, epithelial tissue damage or dysfunction, herpes simplex, post-herpetic neuralgia, disturbances of visceral motility at respiratory, genitourinary, gastrointestinal or vascular regions, wounds, burns, allergic skin reactions, pruritis, vitiligo, general gastrointestinal disorders, colitis, gastric ulceration, duodenal ulcers, and bronchial disorders.

As used herein, “treatment” or “treating” is an approach for obtaining beneficial or desired clinical results. For purposes of this invention, beneficial or desired clinical results include, but are not limited to, one or more of the following: improvement or alleviation of any aspect of pain and/or inflammation, including acute, chronic, inflammatory, neuropathic, or post-surgical pain. For purposes of this invention, beneficial or desired clinical results include, but are not limited to, one or more of the following: including lessening severity, alleviation of one or more symptoms associated with pain and/or inflammation including any aspect of pain and/or inflammation (such as shortening duration of pain and/or inflammation, and/or reduction of pain sensitivity or sensation).

Such pharmaceutical compositions or medicaments may be for, but not limited to, administration by injection. In certain embodiments, the invention encompasses pharmaceutical compositions comprising effective amounts of at least one B1 peptide antagonist (released at a rate and amounts effective to prevent, ameliorate, or abolish pain or any of the B1 mediated medical conditions discussed herein) incorporated within a polymeric matrix. Additionally, such compositions may be further formulated together with other pharmaceutically acceptable diluents, excipients, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimerosol, benzyl alcohol) and bulking substances (e.g., lactose, mannitol). See, for example, Remington's Pharmaceutical Sciences, 18th Edition., Mack Publishing Co., Easton, Pa., pages 1435-1712 (1990), which is herein incorporated by reference. The compositions may be prepared in liquid form, or as a dried powder (such as lyophilized form).

As used herein, the phrases “sustained delivery” or “sustained release” are used interchangeably herein and in reference to an active agent is intended to refer to a release of the active agent from a sustained delivery composition that is longer than that time period during which a therapeutically significant amount of the active agent would be available following direct administration of a solution of the active agent. The resulting in vivo pharmacokinetic (PK) profile of an active agent from a sustained delivery composition is also much more consistent (maintained in a desired window) than the profile observed following administration of the active agent in solution. Sustained delivery can be continuous or discontinuous, and/or linear or non-linear. This can be accomplished using one or more types of polymer compositions, drug loadings, inclusion of excipients or degradation enhancers, or other modifiers, administered alone, in combination or sequentially to produce the desired effect. Zero order or linear release is generally construed to mean that the amount of the bioactive molecule released over time remains relatively constant as a function of amount/unit time during the desired time frame. Multi-phasic is generally construed to mean that release occurs in more than one “burst”. Sustained delivery of the agent can be demonstrated by, for example, the continued prophylactic, therapeutic or diagnostic effect of the active agent over time. Additionally (or alternatively), sustained delivery of the active agent may be demonstrated by detecting the presence of the active agent in vivo over time. In certain embodiments, the sustained delivery is provided for between about 3 days and about 21 days. In other embodiments, in conjunction with the above and below embodiments, the sustained delivery is between about 3 and about 14 days, between about 3 and about 10 days, between about 3 and about 7 days, between about 3 and about 5 days, and about 3 days.

Accordingly, the present invention is directed to the production, composition, and use of sustained delivery compositions that provide prophylactically, therapeutically, and/or diagnostically effective blood-levels of at least one active agent at a desirable rate and duration of between about 3 days and about 21 days.

One exemplary aspect of the present invention may include sustained delivery compositions that modulate the release of at least one active agent incorporated therein, and methods of preparation and use thereof, are disclosed. The compositions include a biodegradable and/or biocompatible polymeric matrix; at least one active agent dissolved and/or dispersed within the polymeric matrix; and a carbohydrate component which is separately dispersed within the polymeric matrix. The carbohydrate component modulates the release of any incorporated active agents from the polymeric matrix at a desired rate and for a period of time to provide for desired blood-levels of the agent or agents for up to about twenty-one.

As used herein, the term ‘about’ is meant to reflect a variability of up to 20% of the enumerated value, whether it is a duration of time as described immediately above, or is for another value.

As used herein, “modulated release”, “accelerated sustained release”, and “accelerated sustained delivery” are used interchangeably and are intended to refer to the change in the release characteristics of an incorporated active agent from a biodegradable and/or biocompatible polymeric matrix containing a dispersed carbohydrate component that is separate from the incorporated active agent relative to a polymeric matrix that does not include the separately dispersed carbohydrate component. Release characteristics include the burst, subsequent agent release levels, the amount of active agent released, and/or the extent of the release period. The release characteristics may be modified by selecting the type and concentration of the carbohydrate component that is dispersed in the polymeric matrix. In addition, the particle size of dispersed carbohydrate component can be selected to modify the release characteristics. In another embodiment, in conjunction with the above and below embodiments, the particle size of the separately dispersed carbohydrate may be from about 10 μm to about 1 μm, 8 μm to about 2μ, 5 μm to about 2 μm, or approximately 2 μm.

### Polymer Selection

Any biocompatible polymer can be used. As used herein, a polymer or polymeric matrix is biocompatible if the polymer and any degradation products of the polymer, are non-toxic to the recipient and also present no significant deleterious effects on the body of the recipient. The biocompatible polymers can be biodegradable polymers, or non-biodegradable polymers, or copolymers and blends thereof.

As used herein, the term “bioerodible” or “biodegradable”, as used herein, refer to polymers that are capable of degrading or eroding to form smaller chemical species over a period of time dissolve or degrade within a period that is acceptable in the desired application (usually in vivo therapy), typically less than about five years, and more preferably less than about one year, once exposed to a physiological solution of pH between about 6-8 and at a temperature of between about 25° C.-38° C.

Examples of suitable biocompatible, biodegradable polymers include poly(lactide)s, poly(glycolide)s, poly(lactide-co-glycolide)s, poly(lactic acid)s, poly(glycolic acid)s, poly(lactic acid-co-glycolic acid)s, polyanhydrides, polyorthoesters, polyetheresters, polycaprolactone, polyesteramides, and copolymers and blends thereof. Preferred polymers include poly(hydroxy acids), especially poly(lactic acid-co-glycolic acid) (“PLGA”) that degrade by hydrolysis following exposure to the aqueous environment of the body. The polymer is then hydrolyzed to yield lactic and glycolic acid monomers, which are normal byproducts of cellular metabolism. The rate of polymer disintegration can vary from several weeks to periods of greater than one year, depending on several factors including polymer molecular weight, ratio of lactide to glycolide monomers in the polymer chain, and stereoregularity of the monomer subunits (mixtures of L and D stereoisomers disrupt the polymer crystallinity enhancing polymer breakdown). Poly(dl,lactide-co-glycolide) type polymers (PLGA, Resomer RG502H, RG502, RG503H, RG503, RG752, R202, R202H) are commercially available from Boehringer Ingelheim (B.I.) Chemicals, Inc. (Petersburg, Va.). Various other suitable polymers are readily commercially available as well.

The poly(lactide-co-glycolide) (hereinafter “PLG”) can have a lactide:glycolide ratio, for example, of about 10:90, 25:75, 50:50, 75:25 or 90:10. In a preferred embodiment of the invention, the lactide:glycolide ratio of the poly(lactide-co-glycolide) copolymer is 50:50. In certain embodiment, the end groups of the poly (lactide-co-glycolide) are in the methyl ester form. In other embodiments, the end groups of the poly(lactide-co-glycolide) polymer are in the acid form. In further embodiments, the ester form and acid form of the poly(lactide-co-glycolide) can be blended at a suitable ratio. For example, from about 10% of either the ester form or acid form 5 to about 90% of the acid form or ester form, respectively. Preferably, the sustained release composition releases its encapsulated active agent over a period of at least 3 days in humans.

Suitable non-biodegradable polymers include polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, blends and copolymers thereof.

The end-groups of the polymers can be blocked, unblocked, or a blend of blocked and unblocked polymers. A blocked polyester is as classically defined in the art, specifically having blocked carboxyl end groups. Generally, the blocking group is derived from the initiator of the polymerization and is typically an alkyl group. Suitable blocking groups include alkyl groups. Preferably, the end-groups of the polymers are unblocked so as to facilitate release of one or more incorporated agents for a duration of up to about twenty-one or less. An unblocked polyester is as classically defined in the art, specifically having free carboxyl end groups. Acceptable molecular weights for the biocompatible and/or biodegradable polymers can be determined by a person of ordinary skill in the art of taking into consideration factors such as the desired polymer degradation rate, physical properties such as mechanical strength, and rate of dissolution of polymer in solvent. Typically, an acceptable range of molecular weight (Mw) is between about 1,000 and about 200,000 Daltons (Da), between about 2,000 Da and about 50,000 Da, between about 2,000 Da and about 20,000 Da, between about 2,000 Da and about 12,000 Da or between about 5,000 Da and about 12,000 Da, for example. The polymer may be, for example, a copolymer such as PLGA with a lactide:glycolide ratio of about 1:1 and a molecular weight between about 5,000 Da and about 20,000 Da.

In another embodiment, in conjunction with the above and below embodiments, the polymer may comprise low-molecular weight polymers. Preferred low molecular weight polymers include those described in and manufactured in accordance with U.S. patent application Ser. No. 11/114,473, filed Apr. 25, 2005 and entitled “Low Molecular Weight Polymers” which was published on Dec. 8, 2005 as U.S. Patent Application Publication No. 2005/0271722. Even more preferred low molecular polymers include the polylactic acid (PLA) polymers described in and manufactured in accordance with U.S. patent application Ser. No. 11/114,473, filed Apr. 25, 2005 and entitled “Low Molecular Weight Polymers”.

### Active Agent(s) to be Incorporated

As used herein, an “active agent” refers to a substance having utility for modulating biological processes so as to achieve a desired effect in the diagnosis, modulation, prevention or treatment of an existing condition in a living being, such as a medical, agricultural or cosmetic effect. Thus, active agents are generally selected from the broad categories of medicaments, radioisotopes, agricultural products and cosmetics.

In certain embodiments, an active agent of a composition of the invention may be a protein, a peptide, and/or a peptide receptor ligand having a non-natural pseudopeptide or peptidomimetic form. As used herein, the terms “protein” and “peptide” are both understood to include polymers of natural and/or non-natural amino acids linked by amide bonds. Typically, a peptide is composed of between two and about 50 amino acids, more typically between two and about 30 amino acids and even more typically, between two and about 20 amino acids. On the other hand, a protein will typically be composed of more than 50 amino acids. The terms “protein” and “peptide” are further intended to encompass analogues, derivatives, conjugates and/or complexes of the protein or peptide as the case may be. Examples of analogues include peptides or proteins containing one or more non-natural amino acids. Examples of derivatives include peptides or proteins containing amino acid side chain(s), peptide backbone, and/or amino- or carboxy-terminus that have been derivatized. Acetylation is a suitable method of derivatization, for example. Examples of conjugates include peptides or proteins conjugated or “fused” to a “vehicle”. The term “vehicle” as used herein refers to a molecule that prevents degradation and/or increases half-life, reduces toxicity, reduces immunogenicity, or increases biological activity of a therapeutic protein or peptide. Suitable vehicles may include another polypeptide such as the Fc region of human IgG1, a water-soluble polymer such as polyethylene glycol (PEG), a lipid, a cholesterol group, a carbohydrate, or an oligosaccharide.

Therefore, in another embodiment, in conjunction with the above and below embodiments, the protein and/or peptide intended for use in the compositions of the present invention may be conjugated with a water soluble vehicle such as polyethylene glycol as described U.S. Patent application Ser. No. 10/972,236, filed Oct. 21, 2004 and entitled “Antagonists of the Bradykinin B1 receptor” (published on Sep. 29, 2005 as U.S. Patent Application Publication No. 2005/0215470) to provide for an even more sustained period of appropriate plasma levels of the peptide upon parenteral administration of a composition of the present invention to a mammal.

Additionally, in another embodiment, in conjunction with the above and below embodiments, the protein and/or peptide intended for use in the compositions of the present invention may be conjugated with a polypeptide vehicle such as the Fc domain of IgG1 as described U.S. patent application Ser. No. 10/666,480, filed Sep. 18, 2003 and entitled “PEPTIDES AND RELATED MOLECULES THAT MODULATE NERVE GROWTH FACTOR ACTIVITY” (published on Jul. 19, 2005 as U.S. Pat. No. 6,919,426) to provide for an even more sustained period of appropriate plasma levels of the peptide upon parenteral administration of a composition of the present invention to a mammal.

In another embodiment, in conjunction with the above and below embodiments, the protein and/or peptide intended for use in the compositions of the present invention may be complexed with a gallic acid ester as described in U.S. patent application Ser. No. 11/114,473, filed Apr. 25, 2005 and entitled “Sustained Release Formulations” (published on Dec. 8, 2005 as U.S. Patent Application Publication No. 2005/0271722) to provide for a more sustained period of appropriate plasma levels of the protein and/or peptide upon parenteral administration of the composition of the present invention to a mammal.

Peptides suitable for formulation according to the invention include but are not limited to enfuvirtide (sold by Trimeris and Roche as Fuzeon®), Angiotensin, Amylin, ACTH, renin substrate, Cecropin A-Melittin amide, Cecropin B, Magainin 1, Renin Inhibitor Peptide, Bombesin, Osteocalcin, Bradykinin, Kallidin, Calcitonin, Cholecystokinin, Corticotropin Releasing Factor, Dynorphin A, Endomorphin, Sarafotoxin, Enkephalin, Exendins, Exenatide, Fibrinopeptide, Galanin, Gastrin, Gastrin Releasing Peptide, Glucagon-Like Peptide, Growth Hormone Releasing Factor, OVA Peptide, Luteinizing Hormone-Releasing Hormone, Atrial Natriuretic Peptide, Melanin Concentrating Hormone, Brain Natriuretic Peptide, Vasonatrin, Neurokinin, Neuromedin, Neuropeptide Y, Neurotensin, Orexin, Oxytocin, Vasopressin, Parathyroid Hormone Peptide, Prolactin Releasing Peptide, Somatostatin, Somatostatin Tumor Inhibiting Analog, Thyrotropin Releasing Hormone, and variants and derivatives thereof (see also, Latham, (1999) Nat. Biotech., 17:755). Additional peptides suitable for formulation according to the present invention include bradykinin peptide antagonists, including, but not limited to, the bradykinin peptide antagonists disclosed or referenced in U.S. patent application Ser. No. 10/972,236 (filed on Oct. 21, 2004) and entitled “ANTAGONISTS OF THE BRADYKININ B1 RECEPTOR” which was published on Sep. 29, 2005 as U.S. Patent Application Publication No. 2005/0215470. For example, embodiments of the present invention may include sustained delivery compositions comprising the B1 peptide antagonists shown in Table 1 hereinbelow.

Additionally (or alternatively), sustained delivery compositions of the present invention may comprise a biocompatible and/or biodegradable polymeric matrix, at least one of the B1 peptide antagonists shown in Table 1 hereinbelow dissolved and/or dispersed within the polymeric matrix, and a carbohydrate component that is separately dispersed within the polymeric matrix. The carbohydrate component modulates the release of the incorporated active agent from the polymeric matrix in a relatively accelerated manner over a period of time between about three and about twenty-one days.

Proteins that can be formulated according to the invention include but are not limited to Flt3 ligand, CD40 ligand, erythropoietin, thrombopoeitin, calcitonin, Fas ligand, ligand for receptor activator of NF-kappa B (RANKL), TNF-related apoptosis-inducing ligand (TRAIL), ORK/Tek, thymic stroma-derived lymphopoietin, granulocyte colony stimulating factor, granulocyte-macrophage colony stimulating factor, mast cell growth factor, stem cell growth factor, epidermal growth factor, RANTES, growth hormone, insulin, insulinotropin, insulin-like growth factors, parathyroid hormone, nerve growth factors, glucagon, interleukins 1 through 18, colony stimulating factors, lymphotoxin-13, tumor necrosis factor, leukemia inhibitory factor, oncostatin-M, and various ligands for cell surface molecules Elk and Hek (such as the ligands for eph-related kinases, or LERKS).

The peptides described herein may be prepared using any method known in the art, for example recombinant or standard solid-phase peptide synthesis techniques (see, e.g., Sambrook, et al., Molecular Cloning: A Laboratory Manual, 2d Ed., Cold Spring Harbor (1989)) and preferably, an automated or semiautomated peptide synthesizer.

The proteins described herein may be prepared using any method known in the art, for example, recombinant protein expression techniques described in Human Cytokines: Handbook for Basic and Clinical Research, Vol. II (Aggarwal and Gutterman, Eds. Blackwell Sciences, Cambridge Mass., 1998); Growth Factors: A Practical Approach (McKay and Leigh, Eds. Oxford University Press Inc., New York, 1993) and The Cytokine Handbook (A W Thompson, ed.; Academic Press, San Diego Calif.; 1991).

Receptors for any of the aforementioned proteins can also be formulated according to the invention, provided that they are soluble portions of the molecule suitable for administration to a subject. Examples include the receptors for both forms of tumor necrosis factor receptor (referred to as p55 and p75), Interleukin-1 receptors (type 1 and 2), Interleukin-4 receptor, Interleukin-15 receptor, Interleukin-17 receptor, Interleukin-18 receptor, granulocyte-macrophage colony stimulating factor receptor, granulocyte colony stimulating factor receptor, receptors for oncostatin-M and leukemia inhibitory factor, receptor activator of NF-kappa B (RANK), receptors for TRAIL, and receptors that comprise death domains, such as Fas or Apoptosis-Inducing Receptor (AIR). A particularly preferred receptor is a soluble form of the IL-1 receptor type II; such proteins are described in U.S. Pat. No. 5,767,064.

Other proteins that can be formulated according to the invention include soluble variants of cluster of differentiation antigens (referred to as CD proteins), for example, those disclosed in Leukocyte Typing VI (Proceedings of the VIth International Workshop and Conference; Kishimoto, Kikutani et al., Eds. Kobe, Japan, 1996), or CD molecules disclosed in subsequent workshops. Examples of such molecules include CD27, CD30, CD39, CD40; and ligands thereto (CD27 ligand, CD30 ligand and CD40 ligand). Several of these are members of the TNF receptor family, which also includes 41BB and OX40; the ligands are often members of the TNF family (as are 41BB ligand and OX40 ligand); accordingly, members of the TNF and TNFR families can also be produced using the present invention.

Enzymatically active proteins can also be formulated according to the invention. Examples include metalloproteinase-disintegrin family members, various kinases, glucocerebrosidase, alpha-galactosidase A, superoxide dismutase, tissue plasminogen activator, Factor VIII, Factor IX, apolipoprotein E, apolipoprotein A-1, globins, an IL-2 antagonist, alpha-1 antitrypsin, TNF-alpha Converting Enzyme, and numerous other enzymes. Ligands for enzymatically active proteins can also be formulated by applying the instant invention.

The inventive compositions and methods are also useful for formulation of other types of proteins, including immunoglobulin molecules or portions thereof, and chimeric antibodies (i.e., an antibody having a human constant region couples to a murine antigen binding region) or fragments thereof Numerous techniques are known by which DNA encoding immunoglobulin molecules can be manipulated to yield DNAs capable of encoding recombinant proteins such as single chain antibodies, antibodies with enhanced affinity, or other antibody-based proteins (see, for example, Larrick et al., 1989, Biotechnology 7:934-938; Reichmann et al., 1988, Nature 332:323-327; Roberts et al., 1987, Nature 328:731-734; Verhoeyen et al., 1988, Science 239:1534-1536; Chaudhary et al., 1989, Nature 339:394-397). The term humanized antibody also encompasses single chain antibodies. See, e.g., Cabilly et al., U.S. Pat. No. 4,816,567; Cabilly et al, European Patent No. 0125023 B1; Boss et al, U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al, EP 0 519 596 A1. For example, the invention can also be used to formulate human antibodies, humanized antibodies, or fragments thereof that immunospecifically recognize specific cellular targets, e.g., any of the aforementioned proteins, the human EGF receptor, the her-2/neu antigen, the CEA antigen, Prostate Specific Membrane Antigen (PSMA), CD5, CD11a, CD18, NGF, CD20, CD45, Ep-cam, other cancer cell surface molecules, TNF-alpha, TGF-beta1, VEGF, other cytokines, alpha 4 beta 7 integrin, IgEs, viral proteins (for example, cytomegalovirus), etc., to name just a few.

Various fusion proteins can also be formulated according to the invention. A fusion protein is a protein, or domain of a protein (e.g., a soluble extracellular domain) fused to a heterologous protein or peptide. Examples of such fusion proteins include proteins expressed as a fusion with a portion of an immunoglobulin molecule, proteins expressed as fusion proteins with a zipper moiety, and novel polyfunctional proteins such as a fusion proteins of a cytokine and a growth factor (i.e., GM-CSF and IL-3, MGF and IL-3). WO 93/08207 and WO 96/40918 describe the preparation of various soluble oligomeric forms of a molecule referred to as CD40L, including an immunoglobulin fusion protein and a zipper fusion protein, respectively; the techniques discussed therein are applicable to other proteins. Another fusion protein is a recombinant TNFR:Fc, also known as “etanercept.” Etanercept is a dimer of two molecules of the extracellular portion of the p75 TNF alpha receptor, each molecule consisting of a 235 amino acid TNFR-derived protein that is fused to a 232 amino acid Fc portion of human IgG1. In fact, any of the previously described molecules can be expressed as a fusion protein including but not limited to the extracellular domain of a cellular receptor molecule, an enzyme, a hormone, a cytokine, a portion of an immunoglobulin molecule, a zipper domain, and an epitope.

The active agents used in connection with the methods and compositions of the invention may also include non-protein or non-peptide active agents. Exemplary non-peptide and non-protein active agents include the following non-limiting categories of active agents: (a) nucleic acids including, but not limited to, anti-sense molecules, short interfering RNAs, aptamers, and/or vectors comprising them; (b) carbohydrates and polysaccharides; (c) viruses and virus particles; (d) organic or inorganic natural or synthetic compounds; (e) conjugates or complexes of (a)-(d); and mixtures if (a)-(e). A further description of these and other active agents that can be used in accordance with the methods and compositions of the present invention are described in U.S. Pat. Nos. 5,482,706, 5,514,670, and 4,357,259.

Additionally, active agents which can be used in connection with the methods and compositions of the invention include, but are not limited to, the following active agents: antianginas, antiarrhythmics, antiasthmatic agents, antibiotics, anticholesterol agents, antidiabetics, antifungals, antihistamines, antihypertensives, antiparasitics, antineoplastics, antiinflammatory agents, cardiac glycosides, herbicides, hormones, immunomodulators, monoclonal antibodies, neurotransmitters, pesticides, radio contrasts, radionuclides, sedatives, steroids, analgesics, vaccines, vasopressors, anesthetics, antigens, receptor ligands, nucleic acids, such as antisense molecules, short interfering RNAs, and/or vectors comprising them, antibiotics, steroids, decongestants, neuroactive agents, anesthetics and sedatives, hematopoietics, antiinfective agents, antidementia agents, antiviral agents, antitumoral agents, antipyretics, analgesics, antiulcer agents, antiallergic agents, antidepressants, decongestants, psychotropic agents, cardiotonics, antiarrythmic agents, vasodilators, antihypertensive agents such as hypotensive diuretics, antidiabetic agents, and anticoagulants.

Active agents may include cytokines, growth factors, factors acting on the cardiovascular system, factors acting on the central and peripheral nervous systems, factors acting on humoral electrolytes and hemal organic substances, factors acting on bone and skeleton, factors acting on the gastrointestinal system, factors acting on the immune system, factors acting on the respiratory system, factors acting on the genital organs, and enzymes.

Exemplary hormones include insulin, growth hormone, parathyroid hormone, luteinizing hormone-releasing hormone (LH-RH), adrenocorticotropic hormone (ACTH), amylin, oxytocin, luteinizing hormone, (D-Tryp6)-LHRH, nafarelin acetate, leuprolide acetate, follicle stimulating hormone (FSH), glucagon, prostaglandins and other factors acting on the genital organs and their derivatives, analogs and congeners. As analogs of the LH-RH, such known substances include those described in U.S. Pat. Nos. 4,008,209, 4,086,219, 4,124,577, 4,317,815, and 5,110,904.

Exemplary antibiotics include tetracycline, aminoglycosides, penicillins, cephalosporins, sulfonamide drugs, chloramphenicol sodium succinate, erythromycin, vancomycin, lincomycin, clindamycin, nystatin, amphotericin B, amantidine, idoxuridine, p-amino salicyclic acid, isoniazid, rifampin, antinomycin D, mithramycin, daunomycin, adriamycin, bleomycin, vinblastine, vincristine, procarbazine, imidazole carboxamide.

Exemplary hematopoietic or thrombopoietic factors include, erythropoietins, granulocyte colony stimulating factor (G-CSF), granulocyte-macrophage stimulating factor (GM-CSF) and macrophage colony stimulating factor (M-CSF), leukocyte proliferation factor preparation (Leucoprol, Morinaga Milk), thrombopoietin, platelet proliferation stimulating factor, megakaryocyte proliferation (stimulating) factor, and factor VIII. Exemplary antidementia agents include selegelene. Exemplary antiviral agents include amantidine and protease inhibitors. Exemplary antitumoral agents include doxorubicin, Daunorubicin, taxol, and methotrexate. Exemplary antipyretics and analgesics include aspirin, Motrin, Ibuprofin, naprosyn, Indocin, and acetaminophen. Exemplary antiinflammatory agents include NSAIDS, aspirin, steroids, dexamethasone, hydrocortisone, prednisolone, and Diclofenac Na. Exemplary antiulcer agents include famotidine, cimetidine, nizatidine, ranitidine, and sucralfate. Exemplary antiallergic agents include antihistamines, diphenydramine, loratadine, and chlorpheniramine. Exemplary antidepressants and psychotropic agents include lithium, amitryptaline, olanzapine, tricyclic antidepressants, fluoxetine, prozac, and paroxetine. Exemplary cardiotonics include digoxin. Exemplary antiarrythmic agents include metoprolol and procainamide. Exemplary vasodilators include nitroglycerin, nifedipine, and Isosorbide dinitrate. Exemplary diuretics include hydrochlorothiazide and furosemide. Exemplary antihypertensive agents include captopril, nifedipine, and atenolol. Exemplary antidiabetic agents include glucozide, chloropropamide, metformin, and insulin. Exemplary anticoagulants include warfarin, heparin, and Hirudin. Exemplary cholesterol lowering agents include lovastatin, cholestyamine, and clofibrate. Exemplary therapeutic agents for treating osteoporosis and other factors acting on bone and skeleton include calcium, alendronate, bone GLA peptide, parathyroid hormone and its active fragments (osteostatin, Endocrinology 129, 324, 1991), histone H4-related bone formation and proliferation peptide (OGP, The EMBO Journal 11, 1867, 1992) and their muteins, derivatives and analogs thereof. Exemplary enzymes and enzyme cofactors include: pancrease, L-asparaginase, hyaluronidase, chymotrypsin, trypsin, tPA, streptokinase, urokinase, pancreatin, collagenase, trypsinogen, chymotrypsinogen, plasminogen, streptokinase, adenyl cyclase, and superoxide dismutase (SOD). Exemplary vaccines include Hepatitis B, MMR (measles, mumps, and rubella), and Polio vaccines. Exemplary immunological adjuvants include: Freunds adjuvant, muramyl dipeptides, concanavalin A, BCG, and levamisole. Exemplary cytokines include lymphokines, monokines, hematopoietic factors and so on. Lymphokines and cytokines useful in the practice of the invention include interferons (e.g., interferon-alpha, -beta and -gamma ), interleukins (e.g. interleukin 2 through 18) and so on. Monokines useful in the practice of the invention include interleukin-1, tumor necrosis factors (e.g. TNF- alpha and -beta ), malignant leukocyte inhibitory factor (LIF). Exemplary growth factors include nerve growth factors (NGF, NGF-2/NT-3), epidermal growth factor (EGF), fibroblast growth factor (FGF), insulin-like growth factor (IGF), transforming growth factor (TGF), platelet-derived cell growth factor (PDGF), hepatocyte growth factor (HGF), glial cell line-derived neurotrophic factor (GDNF), neurturin, artemin, and persephin. Exemplary factors acting on the cardiovascular system include factors which control blood pressure, arteriosclerosis, etc., such as endothelins, endothelin inhibitors, endothelin antagonists described in EP 436189, 457195, 496452 and 528312, JP [Laid Open] No. H-3-94692/1991 and 130299/1991, endothelin producing enzyme inhibitors vasopressin, renin, angiotensin I, angiotensin II, angiotensin III, angiotensin I inhibitor, angiotensin II receptor antagonist, atrial naturiuretic peptide (ANP), antiarrythmic peptide and so on. Exemplary factors acting on the central and peripheral nervous systems include opioid peptides (e.g. enkephalins, endorphins), neurotropic factor (NTF), calcitonin gene-related peptide (CGRP), thyroid hormone releasing hormone (TRH), salts and derivatives of TRH [JP [Laid Open] No. 50-121273/1975 (U.S. Pat. No. 3,959,247, JP [Laid Open] No. 52-116465/1977 (U.S. Pat. No. 4,100,152)], neurotensin and so on. Exemplary factors acting on the gastrointestinal system include secretin and gastrin. Exemplary factors acting on humoral electrolytes and hemal organic substances include factors which control hemaglutination, plasma cholesterol level or metal ion concentrations, such as calcitonin, apoprotein E and hirudin. Laminin and intercellular adhesion molecule 1 (ICAM 1) represent exemplary cell adhesion factors. Exemplary factors acting on the kidney and urinary tract include substances which regulate the function of the kidney, such as brain-derived naturiuretic peptide (BNP), urotensin and so on. Exemplary factors which act on the sense organs include factors which control the sensitivity of the various organs, such as substance P. Exemplary factors acting on the immune system include factors which control inflammation and malignant neoplasms and factors which attack infective microorganisms, such as chemotactic peptides and bradykinins. Exemplary factors acting on the respiratory system include factors associated with asthmatic responses. Also included are naturally occurring, chemically synthesized or recombinant peptides which may act as antagonists to any of the proteins or receptors for the proteins mentioned herein. Also included are naturally occurring, chemically synthesized or recombinant peptides or proteins which may act as antigens, such as cedar pollen and ragweed pollen. These factors are administered, either independently, coupled to haptens, or together with an adjuvant, in the formulations according to the present invention.

TABLE 1
B1 Peptide Antagonists

SEQ
ID
NO:

| | | | | | | | |
|---|---|---|---|---|---|---|---|
| 1 | | | Lys | Arg Pro Pro Gly Phe | Ser Pro | Leu | |
| 2 | | Lys | Lys | Arg Pro Hyp Gly Igl | Ser Digl | Oic | |
| 3 | Gun | | DArg | Arg Pro Hyp Gly Thi | Ser Digl | Oic | |
| 4 | Gun | | DArg | Arg Pro Hyp Gly Thi | Ser Diglb | Oic | |
| 5 | | | DArg | Arg Pro Hyp Gly Thi | Ser DTic | Oic | Arg |
| 6 | | | DArg | Arg Pro Hyp Gly Thi | Ser DTic | Oic | Arg |
| 7 | | | DArg | Arg Pro Hyp Gly Thi | Ser DTic | Oic | Arg |
| 8 | | | DArg | Arg Pro Hyp Gly Thi | Ser DTic | Oic | |
| 9 | | | DArg | Arg Pro Hyp Gly Thi | Ser DHpe | Oic | Arg |
| 10 | Ac | Lys | Lys | Arg Pro Pro Gly Me-Phe | Ser D-β-NaI | Ile | |
| 11 | | | DArg | Arg Pro Hyp Gly Igl | Ser DIgl | Oic | Arg |
| 12 | | Lys | Lys | Arg Pro Hyp Gly Igl | Ser DIgl | Oic | |
| 13 | | Lys | Lys | Arg Pro Hyp Gly Cpg | Ser DTic | Cpg | |
| 14 | | | DArg | Arg Pro Hyp Gly Igl | Ser Df5f | Igl | Arg |
| 15 | | DOrn | Lys | Arg Pro Hyp Gly Cpg | Ser DTic | Cpg | |
| 16 | | DOrn | Lys | Arg Pro Thz Gly Cpg | Ser DTic | Cpg | |
| 17 | | 3Pal | Lys | Arg Pro Hyp Gly Cpg | Ser DTic | Cpg | |
| 18 | | 4Pal | Lys | Arg Pro Hyp Gly Cpg | Ser DTic | Cpg | |
| 19 | | | Cha | Arg Pro Hyp Gly Cpg | Ser DTic | Cpg | |
| 20 | | | 2-Nal | Arg Pro Hyp Gly Cpg | Ser DTic | Cpg | |
| 21 | | | Lys | Arg Pro Hyp Gly Cpg | Ser DTic | Cpg | |
| 22 | | DLys | Lys | Arg Pro Hyp Gly Cpg | Ser DTic | Cpg | |
| 23 | | Lys | DOrn | Arg Pro Hyp Gly Cpg | Ser DTic | Cpg | |
| 24 | | Lys | Cha | Arg Pro Hyp Gly Cpg | Ser DTic | Cpg | |
| 25 | | Lys | Abu | Arg Pro Hyp Gly Cpg | Ser DTic | Cpg | |
| 26 | | Lys | 2-Nal | Arg Pro Hyp Gly Cpg | Ser DTic | Cpg | |
| 43 | | D-Dab | Lys | Arg Pro Hyp Gly Cpg | Ser DTic | Cpg | |
| 44 | Ac | D-Dab | Lys | Arg Pro Hyp Gly Cpg | Ser DTic | Cpg | |
| 45 | | DOrn | Lys | Arg Pro Hyp Gly Cpg | Ser DTic | Cpg | |
| 46 | Ac | DOrn | Lys | Arg Pro Hyp Gly Cpg | Ser DTic | Cpg | |
| 47 | | D-3′Pal | Lys | Arg Pro Hyp Gly Cpg | Ser DTic | Cpg | |
| 48 | Ac | D-3′Pal | Lys | Arg Pro Hyp Gly Cpg | Ser DTic | Cpg | |
| 49 | | D-Lys | D-2-Nal | Arg Pro Hyp Gly Cpg | Ser DTic | Cpg | |
| 50 | | Lys | D-2-Nal | Arg Pro Hyp Gly Cpg | Ser DTic | Cpg | |
| 51 | | | DOrn | Arg Oic Pro Gly Me-Phe | Ser D-β-NaI | Ile | |
| 52 | Ac | | DOrn | Arg Oic Pro Gly Me-Phe | Ser D-β-NaI | Ile | |
| 53 | | DOrn | Lys | Arg Oic Pro Gly Me-Phe | Ser D-β-NaI | Ile | |
| 54 | Ac | DOrn | Lys | Arg Oic Pro Gly Me-Phe | Ser D-β-NaI | Ile | |
| 55 | | | Lys | Arg Pro Pro Gly Phe | Ser D-β-NaI | Ile | |
| 56 | Ac | | Lys | Arg Pro Pro Gly Phe | Ser D-β-NaI | Ile | |
| 57 | | | Orn | Arg Oic Pro Gly Me-Phe | Ser D-β-NaI | Ile | |
| 58 | Ac | | Orn | Arg Oic Pro Gly Me-Phe | Ser D-β-NaI | Ile | |
| 59 | | | Lys | Arg Oic Pro Gly Me-Phe | Ser D-β-NaI | Ile | |
| 60 | Ac | | Lys | Arg Oic Pro Gly Me-Phe | Ser D-β-NaI | Ile | |
| 27 | | Cys(Gly)₃ | Lys | Arg Pro Pro Gly Phe | Ser Pro | Leu | |
| 28 | | Cys(Gly)₅ | Lys | Arg Pro Pro Gly Phe | Ser Pro | Leu | |
| 29 | | Cys(Gly)₅ | LysLys | Arg Pro --- Gly Phe | Ser Pro | Leu | |
| 30 | | Cys(Gly)₅ | LysArgLys | Arg Pro Pro Gly Phe | Ser Pro | Leu | |
| 31 | | CysGly(CH2)₆ | Lys | Arg Pro Pro Gly Phe | Ser Pro | Leu | |
| 32 | | Cys(Gly)₅ | LysLys | Arg Pro Pro Gly Me-Phe | Ser D-β-NaI | Ile | |
| 33 | | Cys(Gly)₅ | LysLys | Arg Pro Hyp Gly Cpg | Ser DTic | Cpg | |
| 34 | | Cys(Gly)₇ | LysLys | Arg Pro Hyp Gly Cpg | Ser DTic | Cpg | |
| 35 | | Ac-Cys(Gly)₅ | LysLys | Arg Pro Hyp Gly Cpg | Ser DTic | Cpg | |
| 36 | | | LysLys | Arg Pro Hyp Gly Cpg | Ser DTic | Cpg | |
| 37 | | | Ac-LysLys | Arg Pro Hyp Gly Cpg | Ser DTic | Cpg | |
| 38 | | CysLys | | Arg Pro Pro Gly Phe | Ser Pro | Leu | |
| 39 | | Cys(Gly)₅ | DOrnLys | Arg Pro Hyp Gly Cpg | Ser DTic | Cpg | |
| 40 | | Cys(Gly)₅ | DOrnLys | Arg Pro Thz Gly Cpg | Ser DTic | Cpg | |
| 41 | | Cys(Gly)₅ | LysDOrn | Arg Pro Hyp Gly Cpg | Ser DTic | Cpg | |
| 42 | | (Gly)₅ | LysLys | Arg Pro Hyp Gly Cpg | Ser DTic | Cpg | |

While specific examples of active agents for use in accordance with this invention are mentioned above and below, this does not mean that other agents are excluded from use as an active agent. Active agents may be naturally occurring, recombinant or chemically synthesized substances. As used herein, “active agent” is also intended to encompass inactive agents, as long as the inactive agent is subsequently converted to an active agent as defined above.

As alluded to above, an active agent can be or include a detectable label (e.g., a radioactive, radiopaque, or magnetic agent) that is useful for detecting the presence of and/or identifying the locations of substances, including, but not limited to, the released active agent in vivo. The various types of labels and methods of labeling active agents are well known to those skilled in the art. It will be understood by those skilled in the art that a magnetic substance, such as a metal, is included within the definition of the term label. Several other specific labels or reporter groups are set forth below. For example, the label can be a radiolabel such as, but not restricted to, [32]P, [3] H, [14] C, [35] S, [125]I, or [131] I. A [32]P label can be conjugated to a protein with a conjugating reagent or incorporated into the sequence of a nucleic acid molecule by nick-translation, end-labeling or incorporation of labeled nucleotide. For example, a [3] H, [14] C or [35] S label can be incorporated into a nucleotide sequence by incorporation of a labeled precursor or by chemical modification, whereas an [125] I or [131] I label is generally incorporated into a nucleotide sequence by chemical modification. Detection of a label can be by methods such as scintillation counting, gamma ray spectrometry or autoradiography.

The label can also be a mass or nuclear magnetic resonance (NMR) label such as, for example, [13] C, [15] N, or [19]O. Detection of such a label can be by mass spectrometry or NMR. Dyes, chemiluminescent agents, bioluminescent agents and fluorogens can also be used to label the active agent. Examples of dyes useful for labeling nucleic acids include ethidium bromide, acridine, propidium and other intercalating dyes, and 4′,6′-diamidino-2-phenylindole (DAPI) (Sigma Chemical Company, St. Louis, Mo.) or other nucleic acid stains. Examples of fluorogens include fluorescein and derivatives, phycoerythrin, allo-phycocyanin, phycocyanin, rhodamine, Texas Red or other fluorogens. The fluorogens are generally attached by chemical modification. The dye labels can be detected by a spectrophotometer and the fluorogens can be detected by a fluorescence detector.

An active agent can also be a chromogen (enzyme substrate) or labeled with a chromogen. Alternatively, the active agent may be biotinylated so that it can be utilized in a biotin-avidin reaction, which may also be coupled to a label such as an enzyme or fluorogen. The active agent can be labeled with peroxidase, alkaline phosphatase or other enzymes giving a chromogenic or fluorogenic reaction upon addition of substrate. A label can also be made by incorporating any modified base, amino acid, or precursor containing any label, incorporation of a modified base or amino acid containing a chemical group recognizable by specific antibodies, or by detecting any bound antibody complex by various means including immunofluorescence or immuno-enzymatic reactions. Such labels can be detected using enzyme-linked immunoassays (ELISA) or by detecting a color change with the aid of a spectrophotometer. Active agents also include therapeutic agents that are useful for treating a disease, disorder, or condition.

As mentioned nucleic acid-containing sustained delivery compositions of the present invention are also contemplated. For example, nucleic acid-containing microparticles of the present invention may include: (1) a biocompatible and/or biodegradable polymer (2) a nucleic acid (e.g., plasmid, viral vector, oligonucleotide, RNA, siRNA, antisense and missense nucleic acids); (3) a polycationic polymer (e.g., polylysine); and (4) a separately dispersed carbohydrate. Thus, a method for forming the nucleic acid-containing sustained delivery composition including PLGA microparticles is provided.

A sufficient amount of one or more active agents is incorporated into the polymeric matrices of the compositions of the present invention so that an effective amount of the active agent(s) is released over a predetermined period of time. An effective amount of an active agent can be readily determined by a person of ordinary skill in the art taking into consideration factors such as body weight; age; physical condition; therapeutic, prophylactic, or diagnostic goal desired; type of agent used; type of polymer used; initial burst and subsequent release levels desired; and desired release rate. Typically, the polymeric matrices will be contain between about 0.1% (weight/weight; hereinafter, “(w/w)”) and about 60% (w/w); between about 0.5% (w/w) and about 50% (w/w); between about 5% (w/w) and about 40% (w/w); between about 5% (w/w) and about 20% (w/w); between about 5% (w/w) and about 15% (w/w), between about 5% (w/w) and about 10% (w/w), between about 5% (w/w) and about 10% (w/w), and about 10%, of the active agent. The incorporated active agent(s) may be dissolved in the polymer or dispersed within the polymer in the form of particles, for example, crystalline particles, non-crystalline particles, solid particles, freeze dried particles, spray dried, and lyophilized particles spray dried. The average size of the active agent particles dispersed within the polymer matrix may be between about 1 μm and about 20 μm, between about 2 μm and about 15 μm, between about 3 μm and about 10 μm, between about 4 μm and about 8 μm, or more preferably less than about 5 μm, and even more preferably less than about 3 μm. The particles also may include a stabilizing agent and/or other excipient.

### Carbohydrate Component

A carbohydrate component, as defined herein, is a component containing at least one kind of carbohydrate. A “carbohydrate” as used herein, is a mono-, di-, or tri-saccharide, or a polyol, such as a polysaccharide. Suitable monosaccharides include, but are not limited to glucose, fructose, galactose, and mannose. A “disaccharide” as defined herein is a compound which upon hydrolysis yields two molecules of a monosaccharide. Suitable disaccharides include, but are not limited to sucrose, lactose, maltose, and trehalose. Suitable trisaccharides include, but are not limited to, raffinose and acarbose. In one embodiment, the carbohydrate may be a non-reducing disaccharide. Preferred carbohydrate components include, for instance, trehalose, maltose, glucose, cellulose, and combinations thereof.

The amount of carbohydrate present in the carbohydrate component can range from about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99% to about 99.5% (w/w). In particular embodiments, the amount of carbohydrate present in the carbohydrate component is between about 90% to about 99% (w/w). In other embodiments, the amount of carbohydrate present in the carbohydrate component is between about 95% to about 99% (w/w).

Furthermore, the amount of carbohydrate present in the carbohydrate component of the composition can range from about 0.5%, 1%, 2%, 3%, 4%, 5%, 6%, 7%, 8%, 9%, 10%, 11%, 12%, 13%, 14%, 15%, 16%, 17%, 18%, 19% (w/w) to about 20% (w/w) of the total dry weight of the composition. In particular embodiments, the amount of carbohydrate present in the carbohydrate component of the composition can range from about from about 5% (w/w) to about 10% (w/w) of the total dry weight of the composition. In some embodiments, the amount of carbohydrate present in the carbohydrate component of the composition is about 10% (w/w) of the total dry weight of the composition. Also, in some embodiments of the present invention, the carbohydrate component, as defined herein, may further comprise at least one salt such as NaCl, NaF, KCl, KF, phosphate, sulfate, acetate, and lactate or any combination thereof. However, the total amount of salt in the carbohydrate component of the composition may be less than about 80% (w/w), about 70%, about 60%, about 50%, about 40%, about 30%, about 20% or less than about 10%. In some embodiments, the total amount of salt in the carbohydrate component of the composition is less than about 50%. Suitable concentrations are those that modulate the release of incorporated agents from the polymeric matrix to provide a sustained delivery composition of a targeted release rate and targeted duration. The optimum concentration depends upon various factors such as target release rate, target release duration, polymer, carbohydrates and/or salts in the carbohydrate component and the biologically active agent utilized. In one embodiment, the carbohydrate component is substantially soluble in aqueous solutions, such as PBS, HEPES, or simulated physiological fluids.

“Surfactant” as the term is used herein refers to any substance which can reduce the surface tension between immiscible liquids. Suitable surfactants which can be added to the sustained release composition include, but are not limited to, polymer surfactants, such as nonionic polymer surfactants, for example, poloxamers, polysorbates, polyethylene glycols (PEGs), polyoxyethylene fatty acid esters, polyvinylpyrrolidone and combinations thereof. Examples of poloxamers suitable for use in the invention include poloxamer 407 sold under the trademark PLURONIC® F 127, and poloxamer 188 sold under the trademark PLURONIC® F68, both available from BASF Wyandotte. Examples of polysorbates suitable for use in the invention include polysorbate 20 sold under the trademark TWEEN® 20 and polysorbate 80 sold under the trademark TWEEN® 80. Cationic surfactants, for example, benzalkonium chloride, may also be suitable for use in the invention. In addition, bile salts, such as deoxycholate and glycocholate are suitable as surfactants based on their highly effective nature as detergents. The surfactant can be present in the polymer phase, the carbohydrate component or the active agent component of the compositions. The surfactant can act to modify release of the active agent from the polymer matrix, can act to stabilize the active agent or a combination thereof. Preferred surfactants include sodium caprate, polyvinyl alcohol, sorbitan monooleate (Span 80), polyethylene sorbitan monooleate (Tween 80)(Sigma-Aldrich Chemie GmbH, Steinheim, Germany), sodium raurate, sodium stearate, sodium palmitate, sodium pamoate, sodium caprylate and combinations thereof. In certain embodiments, the carbohydrate component comprises between about 0.5% (w/w) and about 10%, between about 1% and about 5% (w/w), and between about 1% and about 5% (w/w) of sodium caprate.

Initial Burst of Active Agent Release

The drug release from sustained release delivery systems can usually be divided into an initial release (“burst”) phase followed by a slower continuous release phase. The phrases “initial release phrase”, “burst”, “burst phase”, “initial burst”, or variations thereof may be used interchangeably herein. The initial release which often plays an important role in both the therapeutic efficacy and toxicity of formulations, is normally defined as the amount of drug released during the first 24 hours. Depending on the drug, a lower or higher initial release is required in order to initiate a pharmacological effect; an undesirable high initial release may exhaust the encapsulated drug from microparticles too rapidly and even cause toxicity problems. Thus, the proper control of the initial release phase is one of the key issues in the design of controlled delivery systems.

The initial release is commonly attributed to the release of drug located close to the surface of microparticles or to easily accessible drug, for example in the case of highly porous microparticles (Batycky et al. 1997; Cohen et al., 2002, Herrmann and Bodmeier, 1995b, Ravivarapu et al., 2000c). A high porosity correlates with a large surface area and rapid penetration of the release medium and consequently a high initial release.

A popular method for the preparation of microparticles is the solvent evaporation method (Bodmeier and Chen, 1989). The drug is dissolved, dispersed or emulsified into an organic polymer solution. After emulsification of the polymer phase into an external (mostly aqueous) phase, the solvent diffuses into the external phase and evaporates; simultaneously, the external phase (nonsolvent) penetrates into the surface of the polymer droplets. The precipitation kinetics of the polymer droplets determines the microstructure of the solidified microparticles. In general, a rapid polymer precipitation causes the formation of porous microparticles because of a hardening of the droplets with still significant amount of solvent present, while a slower precipitation results in more concentrated polymer droplets and denser microparticles (Schlicher et al., 1997, Graham et al., 1999). Although having the same final composition, different microstructures of the particles with different release profiles can be obtained.

The PLGA precipitation kinetics in an in situ PLGA implant system was examined by McHugh et al. (Graham et al, 1999, Brodbeck et al., 1999a). Parameters leading to a faster PLGA precipitation (e.g., PVP or water addition to the PLGA solution or a decreasing polymer concentration) resulted in more porous implants and a high initial release. In contrast, a slower precipitation resulted in denser sponge-like implant with a low initial release.

### Methods for Preparing the Polymeric Matrix

Another aspect of the present invention relates to methods for the preparation of the novel sustained delivery compositions disclosed herein. For example, one embodiment of the present invention includes compositions that may be prepared by dissolving a biocompatible and/or biodegradable polymer in a solvent to form a polymer solution, and separately dispersing a carbohydrate component and a prophylactic, therapeutic, and/or diagnostic agent within the polymer solution. The polymer solution is then solidified to form a polymeric matrix. At least a significant amount of the carbohydrates is dispersed in the polymeric matrix separately from the incorporated agent. The carbohydrate modulates the release of the incorporated agent from the polymeric matrix in a relatively consistent manner over a period of time up to about thirty days or less.

In some embodiments of the present invention, the polymeric matrix can be prepared by dissolving a suitable polymer in a solvent to form a polymer solution, adding a solution of the active agent to be incorporated, and adding the carbohydrate component to the polymer solution to form a suspension. Addition of the carbohydrate component can be completed before addition of the active agent. For example, the polymer solution and the carbohydrate solution or particles can be mixed by sonication or agitation, while the active agent is incorporated later in the process of forming the polymeric matrix.

In addition, other excipients can be added to the polymer phase to modify the release of the active agent from the sustained release composition. Such excipients include salts, such as sodium chloride.

“Antioxidants” can also be added to the sustained release composition. Suitable antioxidants can include, but are not limited to, methionine, vitamin C, vitamin E and maleic acid. The antioxidant can be present in the stabilized FSH formulation or added in the polymer phase. In a particular embodiment, methionine can be added to reduce the oxidation of the disulfides and methionine residues in FSH.

In those embodiments in which the polymer is insoluble in aqueous solutions and soluble in organic solvents that are immiscible with water, an emulsion can be formed. Emulsions can be formed, for example, by sonicating, agitating, mixing, or homogenizing these solutions.

### Determining the Relevant Amounts of Incorporated Agent and Carbohydrate Component

The amount of a biologically active agent added to the polymer solution can be determined empirically by comparative in vivo tests of polymeric matrices containing different concentrations of at least one carbohydrate component and of at least one biologically active agent. The amount used will vary depending upon the particular agent, the desired effect of the agent at the planned release levels, and the time span over which the agent will be released.

### Types of Delivery Devices

Several types of delivery devices, such as, thin films, rods, pellets, cylinders, discs, implants, and microparticles can be prepared from the polymeric matrix, using methods well known to those of skill in the art. In a preferred embodiment, the method includes forming a modulated release polymeric matrix as a thin film. A suitable carbohydrate component is dissolved in distilled water and sonicated into the polymer solution along with a biologically active agent also dissolved in distilled water. A thin film is then solvent cast from the polymer solution and left to dry overnight. The film is then subjected to high vacuum for a period of 4-6 hours to extract any residual solvent. A microparticle is more preferred. In microparticle compositions intended for administration to a patient by injection, the size of the microparticles should average about 150, 125, 100, 75, 70, 65, 60, 55, 50, 45, 40, 35, 30, 25, or 20 microns in diameter.

According to another aspect of the invention, a syringe-containing a pharmaceutical composition of the present invention is provided. The syringe may contain a single dose of microparticles containing an active agent for treating a condition that is treatable by the sustained delivery of the active agent form the microparticles; and a needle attached to the syringe, wherein the needle has a bore size that is from 14 to 30 gauge. Additionally, the microparticles of the invention can be prepared to have a dimension which permits the delivery of microparticles using a needleless syringe (MediJector, Derata Corporation, Minneapolis, Minn. 55427), thereby eliminating the disposal problems inherent to needles which must be disposed as a biohazard waste product. Thus, according to a particularly preferred aspect of the invention, a needleless syringe containing a pharmaceutical composition comprising one or more doses of microparticles containing an active agent for treating a condition is provided.

In another embodiment, the method includes forming a modulated release system via the spray drying process. Alternately, the method includes forming modulated release polymer microparticles via the solvent removal process. Either method forms microparticles, or microparticles, encapsulating the carbohydrate component and biologically active agent within the system. As used herein, “microparticles” refers to particles having a diameter of preferably less than 1.0 mm, and more preferably between 1.0 and 100.0 microns. Microparticles include microspheres, which are typically solid spherical microparticles. Microparticles also include microcapsules, which are spherical microparticles typically having a core of a different polymer, drug, or composition. As used herein, microparticles are particles having a diameter of less than about one millimeter that include at least one incorporated agent. The microparticles can have a spherical, non-spherical, or irregular shape. Preferably, the microparticles are spherical.

To form microparticles, in particular, a variety of techniques known in the art can be used. These include, for example, single or double emulsion steps followed by solvent removal.

Solvent removal may be accomplished by extraction, evaporation or spray drying among other methods. In the solvent extraction method, the polymer is dissolved in an organic solvent that is at least partially soluble in the extraction solvent such as water. The active agent, either in soluble form or dispersed as fine particles, is then added to the polymer solution, and the mixture is dispersed into an aqueous phase that contains a surface-active agent such as poly(vinyl alcohol). The resulting emulsion is added to a larger volume of water where the organic solvent is removed from the polymer/active agent to form hardened microparticles. In the solvent evaporation method, the polymer is dissolved in a volatile organic solvent. The active agent, either in soluble form or dispersed as fine particles, is then added to the polymer solution, and the mixture is suspended in an aqueous phase that contains a surface-active agent such as poly(vinyl alcohol). The resulting emulsion is stirred until most of the organic solvent evaporates, leaving solid microparticles. In the spray drying method, the polymer is dissolved in a suitable solvent, such as methylene chloride (e.g., 0.04 g/ml). A known amount of active agent is then suspended (if insoluble) or co-dissolved (if soluble) in the polymer solution. The solution or the dispersion is then spray-dried. Microparticles ranging in diameter between one and ten microns can be obtained with a morphology, which depends on the selection of polymer.

The type of solvent used to dissolve the polymer will depend on the type of polymer. Suitable solvents for dissolving the various biodegradeable polymers include polar organic solvents such as methylene chloride, chloroform, acetone, ethyl acetate, tetrahydrofuran, dimethyl sulfoxide, dichloroethane, and hexafluoroisopropanol. Suitable solvents for poly(lactide-co-glycolide) include include dimethysulfoxide, ethyl acetate, methylacetate, methylene chloride, chloroform, hexafluoroisopropanol, acetone, and combinations thereof.

The term “microdroplet” as used herein, refers to a droplet of any morphology which has a dimension less than or equal to about 1,000 microns.

Similarly, the type of solvent used to dissolve any particular active agent will depend on the type and particular characteristics of the active agent(s). Suitable solvents for proteins or peptides, may include, but is not limited to, ethanol, methanol, water, acetonitrile, dimethylformamide, DMSO, and combinations thereof. In one embodiment, particles of a carbohydrate component are pre-dissolved in distilled water and then dispersed within the polymer solution. At least one biologically active agent is added to the polymer solution separately from the addition of the carbohydrate component solution. The biologically active agent can also be dissolved in distilled water, thereby adding to the polymer and carbohydrate component emulsion.

The carbohydrate component and the biologically active agent can be added to the polymer solution sequentially, in reverse order, intermittently, or through separate, concurrent additions. A biologically active agent can be suspended in a solution of a carbohydrate component in a solvent before dissolving the polymer in the solvent.

In another embodiment, the carbohydrate component is incorporated into the polymeric matrix after the matrix has been formed and has already incorporated the active agent. In an alternate embodiment, the protein or active drug added to the polymer solution can be mixed with an excipient, such as at least one stabilizing agent or anti-oxidizing agent, as is known in the art.

Microspheres formed by the solvent evaporation process are not contemplated to be within the microparticles disclosed herein, unless they were left for a very short time to harden. Otherwise, the carbohydrate component would leach out of the system during the fabrication of the system.

In another embodiment, the method includes forming a modulated release polymeric matrix as a rod, cylinder, or any other shape. A polymer solution and carbohydrate component, in dissolved form, are mixed, for example by sonication, until a fine emulsion is produced. The polymer solution is subsequently cast into a mold of the desired shape. The solvent is then removed by means known in the art until a cylinder or other form, with a constant dry weight, is obtained.

In some particular embodiments of the methods for forming B1 peptide antagonist sustained released compositions a poly(lactide-co-glycolide) such as RG502H (B.I. Chemicals, Inc., (Petersburg, Va.)) having an average molecular weight from about 5 kD and 20 kD is dissolved in methylene chloride to form a polymer solution. The polymer solution is added to a solution of peptide component comprising at least one B1 peptide antagonist dissolved in methanol such that the total weight of the B1 peptide antagonists will be between about 1% (w/w) and about 15% (w/w) of the dry weight of the final composition. The polymer solution and the peptide solution are then mixed and added to an amount of spray-dried particles of a carbohydrate component comprising 99% trehalose and 1% sodium caprate. The copolymer/peptide component/carbohydrate component mixture is spray dried or freeze spray-dried and the B1 peptide antagonist microparticle composition is collected. The PLGA microparticles fabricated using methylene chloride and methanol as the co-solvents for the PLGA and the B1 peptide antagonist component, respectively, have a dramatically lower in vivo burst (as defined by maximum plasma concentration, Cmax), as well as, an increase in sustained plasma level of the B1 peptide antagonist when the percentage of methanol in the co-solvent solution is below about 20%, about 15%, about 9%, about 7%, about 5%, about 3%, or about 2%.

In another embodiment, in conjunction with the above and below embodiments, sustained release compositions are provided having desirable burst characteristics. In some embodiments, the average burst release of the active agent may range from about 40%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, or 11% to about 10% when placed in a relevant aqueous environment either in vitro or in vivo. Suitably relevant in vitro aqueous environments include, but are not limited to, blood plasma or Dulbecco's phosphate buffer saline (PBS). Suitably relevant in vivo environments, include, but are not limited to, within the body, for instance, when the composition is administered parentally to a patient.

The compositions described herein can be administered to a human, or other mammal, by parenteral administration including injection subcutaneously, intramuscularly, intraperitoneally, intradermally, intravenously, intraarterially or intrathecally.

The sustained delivery compositions may be administered alone or in combination with other drug therapies as part of a pharmaceutical composition. Such a pharmaceutical composition may include the sustained delivery compositions in combination with any standard physiologically and/or pharmaceutically acceptable carriers that are known in the art. The compositions should be sterile and contain a therapeutically effective amount of the microparticle in a unit of weight or volume suitable for administration to a patient. The term “pharmaceutically-acceptable carrier” as used herein means one or more compatible solid or liquid filler, diluents or encapsulating substances which are suitable for administration into a human or other animal. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy. Pharmaceutically acceptable further means a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, desiccants, bulking agents, propellants, acidifying agents, coating agents, solubilizers, and other materials which are well known in the art. Carrier formulations suitable for oral, subcutaneous, intravenous, intramuscular, etc. administrations can be found in Remington's Pharmaceutical Sciences, Mack Publishing Co., Easton, Pa.

A variety of administration routes are available. The particular mode selected will depend, of course, upon the particular drug selected, the severity of the condition being treated, and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular, or infusion. Oral administration will be preferred for prophylactic treatment because of the convenience to the patient as well as the dosing schedule.

The pharmaceutical compositions may conveniently be presented in unit dosage form and may be prepared by any of the methods well-known in the art of pharmacy. All methods include the step of bringing the microparticle into association with a carrier which constitutes one or more accessory ingredients. In general, the compositions are prepared by uniformly and intimately bringing the sustained delivery compositions into association with a liquid carrier, a finely divided solid carrier, or both, and then, if necessary, shaping the product.

Preparations for parenteral administration include sterile aqueous or non-aqueous solutions, suspensions, and emulsions. Additional examples of solvents include propylene glycol, polyethylene glycol, vegetable oils such as olive oil, and injectable organic esters such as ethyl oleate. Aqueous carriers include water, salts and buffer solutions such as saline and buffered media, alcoholic/aqueous solutions and emulsions or suspensions. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like Preservatives and other additives may also be present such as, for example, antimicrobials, anti-oxidants, chelating agents, and inert gases and the like. In general, the sustained delivery compositions can be administered to the subject (any mammalian recipient) using the same modes of administration that currently are used for microparticle therapy in humans. The sustained delivery compositions are useful for a wide variety of separations, diagnostic, therapeutic, industrial, commercial, cosmetic, and research purposes as discussed in more detail below. For example, for in vivo diagnostic purposes, the sustained delivery compositions can include a macromolecule such as an immunoglobulin or cell receptor labeled with a detectable label. Administration of the labeled microparticle to a patient creates an imaging agent for the diagnosis of a proliferative disorder such as cancer or a tool for the evaluation of the success of a therapeutic agent in reducing the proliferation of a particular adverse cell or organism.

Furthermore, the sustained delivery compositions can be used as adjuvants for vaccine production wherein antigen-containing sustained delivery compositions are injected into a research animal, such as a mouse or rabbit, to trigger an enhanced immune response for the production of antibodies to the antigen.

### In Vitro Diagnostics

In Vitro Assays: The sustained delivery compositions described herein are useful as solid phase particles in an assay, such as an enzyme-linked immunosorbant assay, dot-blot, or Western blot, for the detection of a particular target such as a cell, biomolecule or drug in a biological sample. The sustained delivery compositions designed for this use are composed of affinity molecules specific for the target molecule. For example, the macromolecule is an immunoglobulin, cell receptor or oligonucleotide probe and is bound to a test tube or microtiter plate. For detection or quantitation of a target molecule of interest, a sample is combined with a solution containing the sustained delivery compositions, preferably, microparticles, the macromolecules released by the microparticles react with the target molecule, the microparticles are separated from any non-bound components of the sample, and microparticles containing bound molecules are detected by conventional methods. Fluorescently stained microparticles are particularly well suited for flow cytometry analysis in accordance with methods well known to those skilled in the art.

The microparticles described herein are also useful as visual probes or markers of pathology in a histological sample. The macromolecules of microparticles designed for this use are specific for biomolecules expressed during a particular pathologic condition and are labeled with a detectable label. For example, the macromolecule is an immunoglobulin, cell receptor or oligonucleotide probe specific for an abnormal cell, such as a rapidly proliferating cell, or pathological organism, for example, a virus. For detection of a pathogenic condition, a histological sample is combined with a solution containing the microparticles, the labeled macromolecules on the microparticles are reacted with the target molecule of interest, and bound microparticles are detected by detecting the label in accordance with methods well known to those skilled in the art.

The microparticles described herein are useful as imaging agents for in vivo localization of a particular molecule, cell type or pathologic condition in a manner similar to that described above with regard to the use of the microparticles for histopathology. The macromolecules on microparticles designed for this use are specific for molecules expressed by a particular cell or pathologic organism and are labeled with a detectable label. For example, the macromolecule is an immunoglobulin, cell receptor or oligonucleotide probe specific for a tumor cell or pathological organism, such as a virus.

The microparticles are used to either detect a pathologic condition or to monitor the success of therapy, such as chemotherapy or surgery to ensure that the size of an abnormal tissue tumor has decreased or has been completely excised. For this use, a patient receives an administration of a microparticle solution, preferably intravenously, the labeled macromolecules on the microparticles are given a sufficient amount of time to localize to the affected organ or region of the body, the macromolecule is reacted with a target molecule expressed by the cell or organism under investigation, and bound microparticles are detected by detecting the label by conventional imaging techniques well known to those skilled in the art, such as x-ray.

Sustained delivery compositions comprising antigenic proteins or polysaccharide-protein conjugates capable of provoking an immune response are particularly suitable for use as vaccines. The sustained delivery compositions are also useful as vehicles for gene therapy or the production of “genetic vaccines” when comprising nucleic acids, such as DNA or RNA, that are either incorporated into the DNA of the patient or are transfected into a target cell to produce a desired protein. For example, polynucleotides encoding core proteins of viruses such as influenza or human immunodeficiency virus HIV can be delivered as microparticles for expression of an antigenic protein. The nucleic acid microparticles are delivered to mammalian cells in much the same way as naked DNA is delivered. The desired nucleic acid sequence is inserted into a vector, such as plasmid DNA, with a promoter, such as the SV40 promoter or the cytomegalovirus promoter, and optionally may include a reporter gene, such as beta-galactosidase. The nucleic acid is preferably combined with a carrier protein and/or a cation, such as polylysine, to facilitate particle formation as described above. The microparticles are then administered directly to the patient or are transfected into mammalian cells that are then administered to the patient requiring therapy or prophylaxis. The nucleic acid microparticles may include a substance such as chloroquine, which allows nucleic acids to escape from cytoplasmic compartments into the cytoplasm so that it can be more easily transcribed and translated by the cells. Additionally, the microparticles may be coated with a substance that increases the efficiency of translation or may be coated with a substance to provide cell-specific targeting of the microparticles. The invention will be more fully understood by reference to the following examples. These examples, however, are merely intended to illustrate the embodiments of the invention and are not to be construed to limit the scope of the invention.

The following abbreviations are used:
DMSO—dimethyl sulfoxide
DMF—N,N-dimethylformamide
THF—tetrahydrofuran
Et₂O—diethyl ether
EtOAc—ethyl acetate
MeOH—methyl alcohol
EtOH—ethyl alcohol
MeCN—acetonitrile
MeI—iodomethane
NMP—1-methyl-2-pyrrolidinone
DCM—dichloromethane
DCE—1,2-dichloroethane
TFA—trifuoroacetic acid
sat.—saturated
hr—hour(s)
min.—minute(s)
RT—room temperature
ml and μm—milliliter and micrometer.

### EXAMPLES

### Example 1

### Reduction of MP Duration In Vivo with Salt Containing Porogen

0.7761 g of PLGA polymer (RG502H, B.I. Chemicals, Inc. (Petersburg, Va.)) (Lot #270604-640802), with a number average molecular weight of Mn=4750 g/mol by potential acid end group titration, was dissolved in 7.10 mL of methylene chloride. 0.1230 g of a B1 peptide antagonist having the sequence shown in SEQ ID NO: 15 (Peptide A) was dissolved in 0.8177 mL of MeOH (peptide solution); the polymer solution was subsequently added to this solution. The resulting mixture was vortexed and was added into a second vial containing 0.0998 g of spray dried salt containing porogen particles. The composition of the porogen is 16.2% trehalose, 1.78% KCl, 1.8% KH₂PO₄, 70.3% NaCl, and 10.1% Na₂HPO₄ (salt containing porogen). The salt containing porogen particle size was measured to be d(0.5)˜2.5 μm using the Malvern2000. The resulting suspension was briefly sonicated at <20° C. and subsequently atomized to fabricate microparticles using a spray freeze process essentially as described in Burke, et al.,. Pharm. Res. 21:500-506 (2004). The suspension was atomized over a pool of liquid nitrogen. The liquid nitrogen was allowed to evaporate off, and pentane, chilled to a temperature of −120° C., was added to the still-frozen microparticles. The methylene chloride was then extracted from the resulting mixture. Microparticles were filtered and rinsed with chilled pentane, −120° C. and dried in a lyophilizer to remove residual solvents. The resulting powder was sifted through a 125 μm sieve and the powder identified as Lot #49666-040212A. SEM microscopy revealed spherical microparticles. Microparticles were also characterized for particle size, peptide load, and in vitro release in PBS. Encapsulation efficiency of the peptide, based on the nominal load of 10 wt % peptide was 93%.

Lot #49666-040212A microparticles were suspended in an injection vehicle (25 mM NaH2PO4, 0.9% NaCl, 2.5% carboxymethylcellulose, 0.1% Tween 80, pH 7.4) and was injected subcutaneously into male Sprague-Dawley rats at 10 mg/kg peptide to evaluate the performance as a sustained peptide delivery formulation. Plasma concentration levels of Peptide A in rats for were measurable for 10 days for a PLGA/salt containing porogen-encapsulated microparticle (Lot #49666-040212A). As a comparison, plasma concentration-time profiles are plotted for the solution bolus of Peptide A and a PLGA-encapsulated microparticle of Peptide A (Lot #49666-040311G), which show release profiles for 8 hours and >14 days, respectively.

### Example 2

### Reduction of In Vivo Duration with Salt-Free Porogen

0.4666 g of PLGA polymer (RG502H, BI Chemicals, Inc. Lot #270604-640802) was dissolved in 4.32 mL of methylene chloride. 0.0734 g of Peptide A was dissolved in 0.147 mL of MeOH (peptide solution); the polymer solution was subsequently added to this solution. The resulting mixture was vortexed and was added into a second vial containing 0.06 g of spray dried porogen particles. The composition of the porogen is 99% trehalose and 1% CapricNa (salt-free porogen). The salt free porogen particle size was measured to be d(0.5)˜2.5 μm using the Malvern2000. The resulting suspension was sonicated briefly at <20° C. and subsequently atomized to fabricate microparticles using the spray freeze process. The suspension was atomized over a pool of liquid nitrogen, effectively flash freezing the droplets. The liquid nitrogen was allowed to evaporate off, and pentane, chilled to a temperature of −120° C., was added to the still-frozen microparticles. The methylene chloride was extracted. Microparticles were filtered and rinsed with chilled pentane, −120 C. and dried in a lyophilizer to remove residual solvents. The resulting powder was sifted through a 125 μm sieve and the powder identified as Lot #49666-040420B.

Lot #040323A-F was prepared similarly to Lot #49666-040420B except that the porogen is the salt-containing carbohydrate porogen.

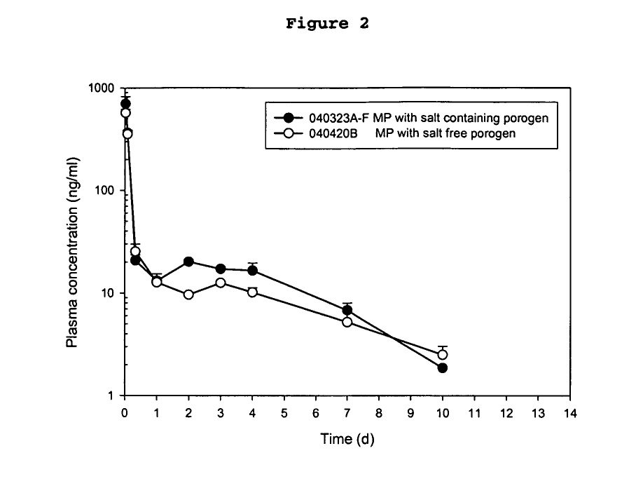

Lot ##49666-040420B microparticles were suspended in an injection vehicle (25 mM NaH2PO4, 0.9% NaCl, 2.5% carboxymethylcellulose, 0.1% Tween 80, pH 7.4) and was injected subcutaneously into male Sprague-Dawley rats at 10 mg/kg peptide to evaluate the performance as a sustained peptide delivery formulation. FIG. 2 shows measurable plasma concentration levels of the active agent in rats for ˜10 days for PLGA/salt free porogen-encapsulated Peptide A microparticle. As a comparison, plasma concentration-time profiles are plotted for PLGA/salt containing porogen-encapsulated Peptide A microparticle (Lot #040323A-F), which show release profiles for 10-14 days. Thus, salt-free porogen excipients are also useful for accelerating the release rate, hence shortening the duration of the microparticle formulation.

### Example 3

### Reduction of MP Duration In Vivo with Salt-Free Porogen

0.7746 g of PLGA polymer (RG502H, B.I. Chemicals, Lot #270604-640802), with a number average molecular weight of Mn=4750 g/mol by potential acid end group titration, was dissolved in 7.20 mL of methylene chloride. 0.1283 g of Peptide A was dissolved in 0.244 mL of MeOH (peptide solution); the polymer solution was subsequently added to this solution. The resulting mixture was vortexed and was added into a second vial containing 0.100 g of porogen. The porogen was fabricated by spray drying a Trehalose w/1% CapricNa solution using a Buchi spray dryer. The salt free porogen particle size was measured to be d(0.5)˜2.5 μm using the Malvern2000. The resulting suspension was sonicated briefly and subsequently atomized to fabricate microparticles using the spray freeze process referenced in Example 1. The suspension was atomized over a pool of liquid nitrogen, effectively flash freezing the droplets. The liquid nitrogen was allowed to evaporate off, and pentane, chilled to a temperature of −120° C., was added to the still-frozen microparticles. The methylene chloride was extracted. Microparticles were filtered and rinsed with chilled pentane, −120° C. and dried in a lyophilizer to remove residual solvents. The resulting powder was sifted through a 125 pm sieve and the powder identified as Lot #040819F.

Lot #040819H was prepared similarly to Lot #040819F except for the removal of the porogen step. 0.873 g of PLGA polymer (RG502H, B.I. Chemicals, Inc., Lot #270604-640802) was dissolved in 8.10 mL of methylene chloride. 0.1267 g of Peptide A was dissolved in 0.276 mL of MeOH; the polymer solution was subsequently added to this solution. The resulting mixture was vortexed and subsequently atomized to fabricate microparticle as above.

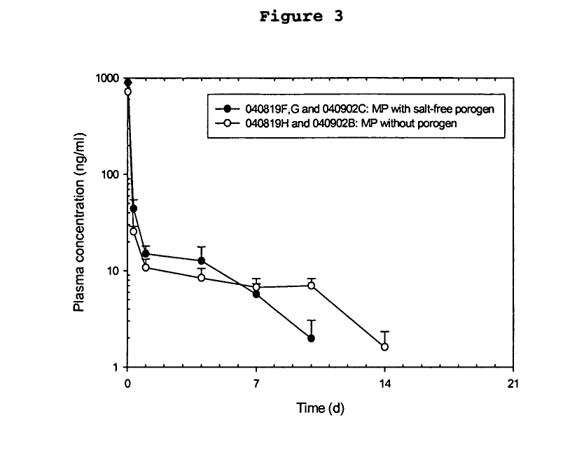

Lot #040819F and 040819H microparticles were respectively suspended in the injection vehicle (25 mM NaH2PO4, 0.9% NaCl, 2.5% carboxymethylcellulose, 0.1% Tween 80, pH 7.4) and were injected subcutaneously into male Sprague-Dawley rats at 10 mg/kg peptide (Study# 103902_{—}09202004) to evaluate the performance as a sustained peptide delivery formulation. FIG. 3 shows measurable Peptide A plasma concentration levels in rats for −10 days for PLGA/salt free porogen-encapsulated Peptide A microparticle as compared to −14 days for PLGA encapsulated Peptide A microparticles. Porogen excipients are useful for accelerating the release rate, hence shortening the duration of the microparticle formulations.

### Example 4

### Reduction of a MP Duration with Salt-Free Porogen; MP Fabricated with a Different Solvent and Polymer Lot

Microparticles were fabricated as in Example 3 with the following differences: 1) polymer lot is 5050DL2A, Medisorb® (Alkermes, Inc.; Cambridge, Mass.) Lot #B2184-5532, with a number average molecular weight of Mn=4750 g/mol by potential acid end group titration and 2) polymer solvent is dichloroethane. MP with and without salt-free porogen were fabricated as described in Example 3 and identified as 040824B and 040824A, respectively.

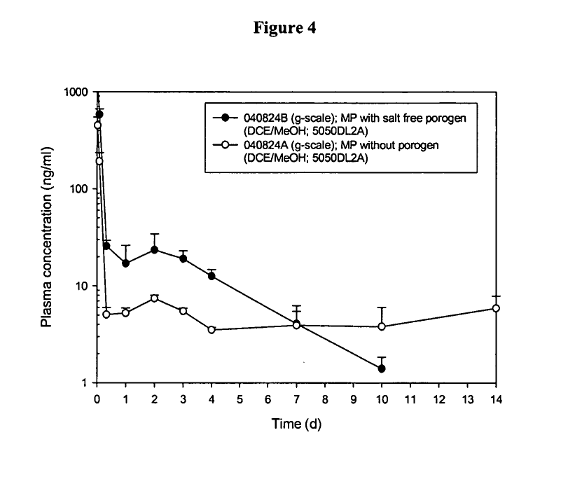

Lot #040824B and 040824A microparticles were respectively suspended in the injection vehicle (25 mM NaH2PO4, 0.9%NaCl, 2.5% carboxymethylcellulose, 0.1% Tween 80, pH 7.4) and were injected subcutaneously into male Sprague-Dawley rats at 10 mg/kg peptide to evaluate the performance as a sustained peptide delivery formulation. Consistent to the findings above, FIG. 4 shows measurable Peptide A plasma concentration levels in rats for ˜10 days for PLGA/salt free porogen-encapsulated Peptide A microparticles as compared to ˜14 days for PLGA encapsulated Peptide A microparticles.

### Example 5

### Demonstration of Accelerated Release and Erosion Rate (Rate of Polymer Disappearance) in Rats

Microparticles with and without porogen were fabricated as described above in Example 3. The porogen utilized included both salt-free and salt-containing forms. Several lots of each microparticle formulation were prepared, resupended in the injection vehicle, and injected subcutaneously into rats at 10 mg/kg peptide. The summary of PK results and necropsy observations from several in vivo studies is displayed in Table 2 below. Table 2 shows that the incorporation of a carbohydrate porogen in the microparticle formulation significantly decreases the percentage of rats with measurable Peptide A plasma concentration level at day 14, thereby demonstrating accelerated release rate and shortening of duration. Furthermore, at day 14, necropsy showed a decrease in incidents of test articles present at the injection site, thereby illustrating increased in vivo erosion rate.

TABLE 2
PK and necropsy findings showing decrease
duration with porogen strategy

| | | % Rats (#) with test |
|---|---|---|
| Microparticle | % Rats (#) with plasma | article present at injection |
| Formulation | level > QL at 14 d | site at 14 d |
| With porogen | 13% (4/30) | 35% (9/26) |
| Without porogen | 89% (8/9) | 57% (4/7) |

### Example 6

### Reduction of MP Duration with Salt Containing Porogen; MP Loaded with an Alternative Drug

0.7043 g of PLGA polymer (RG502H, B.I. Chemicals, Inc., Lot #270604-640802), with a number average molecular weight of Mn=4232 g/mol by potential acid end group titration, was dissolved in 6.5 mL of methylene chloride. 0.35 mL of 0.15 g/ml of a B1 peptide antagonist having the sequence shown in SEQ ID NO:37 (Peptide B) in MeOH was added to the polymer solution. The resulting mixture was vortexed and was added into a second vial containing 0.0835 g salt-containing carbohydrate porogen. The porogen particle size was measured to be d(0.5)˜3 μm using the Malvern2000. The resulting suspension was briefly at <20° C. and subsequently atomized to fabricate microparticles using the spray freeze process. Seven milliliters of suspension was atomized over a pool of liquid nitrogen, effectively flash freezing the droplets. The liquid nitrogen was allowed to evaporate off, and pentane, chilled to a temperature of −120° C., was added to the still-frozen microparticles. The methylene chloride was extracted. Microparticles were filtered and rinsed with chilled pentane, −120° C. and dried in a lyophilizer to remove residual solvents. The resulting powder was sifted through a 125 μm sieve and the powder identified as Lot #43815-030320H. SEM microscopy revealed spherical microparticles (data not shown). Microparticles were characterized for particle size, peptide load, and in vitro release in PBS.

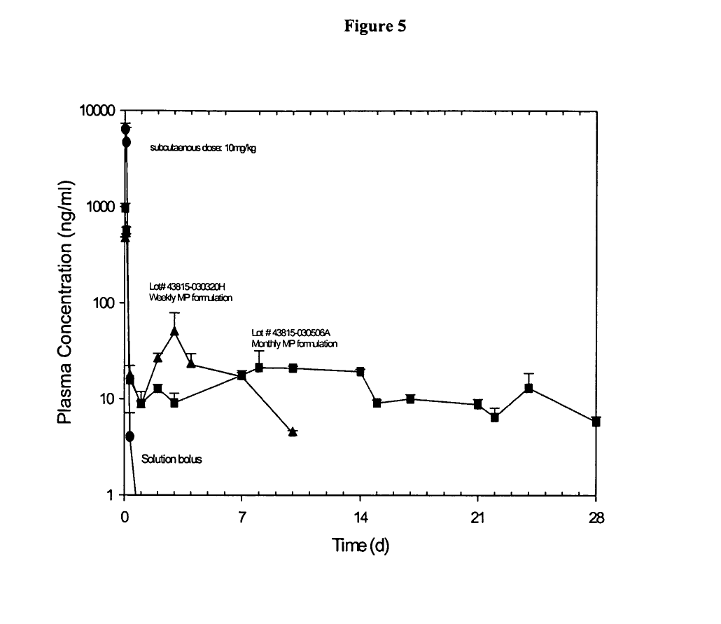

43815-030320H microparticles were suspended in an injection vehicle (25 mM NaH2PO4, 0.9% NaCl, 2.5% carboxymethylcellulose, 0.1% Tween 80, pH 7.4) and was injected subcutaneously into male Sprague-Dawley rats at 10 mg/kg peptide (Study# 102438_{—}03312003) to evaluate the performance as a sustained peptide delivery formulation. FIG. 5 shows measurable Peptide B plasma concentration levels in rats for 10-14 days for PLGA/porogen-encapsulated Peptide B microparticle (Lot #43815-030320H). As a comparison, plasma concentration-time profiles are plotted for the solution bolus of Peptide B and PLGA-encapsulated Peptide B microparticle (Lot #43815-030506A), which show release profiles for 8 hours and a month, respectively.

### Example 7

### Reduction of MP Duration with an Alternative Salt-Free Porogen (Methylcellulose w/5% CapricNa)

0.3887 g of PLGA polymer (RG502H, BI Lot #270604-640802), with a number average molecular weight of Mn=4750 g/mol by potential acid end group titration, was dissolved in 3.60 mL of methylene chloride. 0.0612 g of Peptide A was dissolved in 0.123 mL of MeOH (peptide solution); the polymer solution was subsequently added to this solution. The resulting mixture was vortexed and was added into a second vial containing 0.050 g of porogen. The porogen was fabricated by spray drying Methylcellulose w/5% CapricNa solution using a Buchi spray dryer. The salt free porogen particle size was measured to be d(0.5)˜2.5 μm using the Malvern2000. The resulting suspension was sonicated briefly and subsequently atomized to fabricate microparticles using the spray freeze process. The suspension was atomized over a pool of liquid nitrogen, effectively flash freezing the droplets. The liquid nitrogen was allowed to evaporate off, and pentane, chilled to a temperature of −120° C., was added to the still-frozen microparticles. The methylene chloride was extracted. Microparticles were filtered and rinsed with chilled pentane, −120° C. and dried in a lyophilizer to remove residual solvents. The resulting powder was sifted through a 125 μm sieve and the powder identified as Lot #041014E.

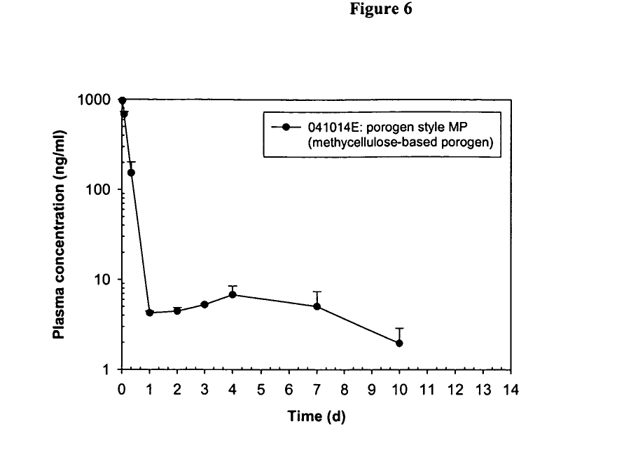

Lot #041014E microparticle was suspended in the injection vehicle (25 mM NaH2PO4, 0.9% NaCl, 2.5% carboxymethylcellulose, 0.1% Tween 80, pH 7.4) and was injected subcutaneously into male Sprague-Dawley rats at 10 mg/kg peptide (Study# 103902_{—}11012004) to evaluate the performance as a sustained peptide delivery formulation. FIG. 6 shows measurable Peptide A plasma concentration levels in rats for −10 days for PLGA/Methylcellulose porogen-encapsulated Peptide A microparticle as previously observed with PLGA/Trehalose porogen-based MP.

### Example 8

### Reduction of a MP Duration with Salt-Free Porogen Fabricated by a Different Process

Microparticles with the composition in Example 3 (5050DL2A polymer, Peptide A, and salt-free porogen) were fabricated by spray drying followed by carbon dioxide extraction (SD), as well as, by spray freeze drying (SF) described in Example 3.

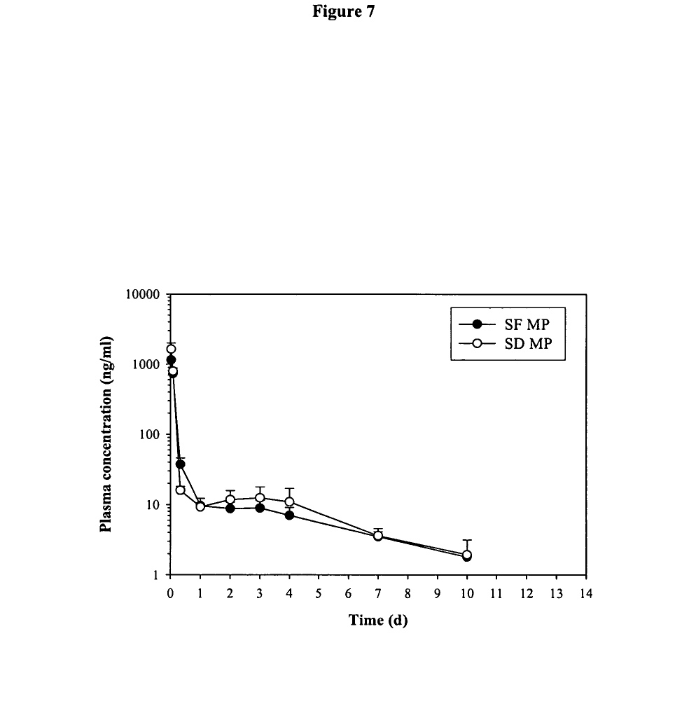

Microparticles fabricated from SD and SF processes respectively were suspended in the injection vehicle and were injected subcutaneously into male Sprague-Dawley rats at 10 mg/kg peptide to evaluate the performance as a sustained peptide delivery formulation. FIG. 7 shows comparable pharmacokinetic profiles with measurable Peptide A plasma concentration levels in rats for ˜10 days for PLGA/salt free porogen microparticles fabricated by both the SD and SF processes. Porogen excipients are useful for accelerating the release rate, hence shortening the duration of the microparticle formulations prepared with a different fabrication process.

### Example 9

### Reduction of MP Burst with Manipulation of Methanol Content in the Fabrication Co-Solvent

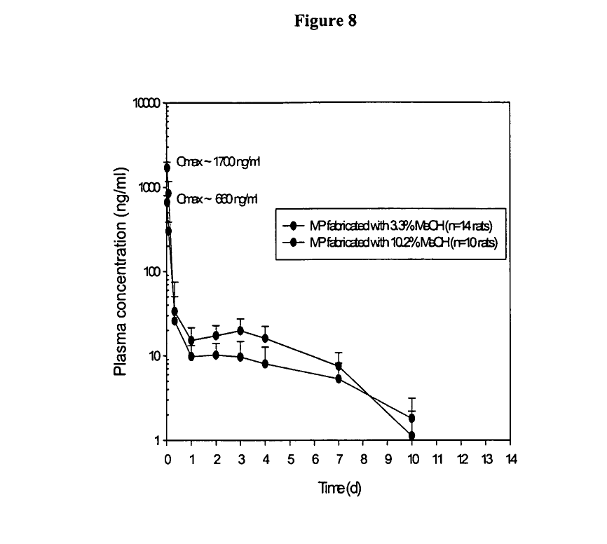

Microparticles with salt-free porogen were fabricated as in Example 3 with the following difference: percentage of methanol in fabrication co-solvent ranged from 3.3 to 10.2%. Microparticles were suspended in the injection vehicle and were injected subcutaneously into male Sprague-Dawley rats at 10 mg/kg peptide to evaluate the performance as a sustained peptide delivery formulation. FIG. 8 shows that microparticles fabricated with low methanol ratio results in a reduction in the in vivo burst (as defined by maximum plasma concentration, Cmax), as well as, an increase in sustained plasma level of Peptide A.

### Example 10

### Increase in Burst with Increase Drug and Porogen Load

Microparticles were fabricated as in Example 3 with the following differences: 1) polymer lot is RG502H Lot #1009848, with a number average molecular weight of Mn=4260 g/mol by potential acid end group titration; 2) Peptide A load varies from 10-15% by weight; and 3) porogen load varies from 0-30% by weight.

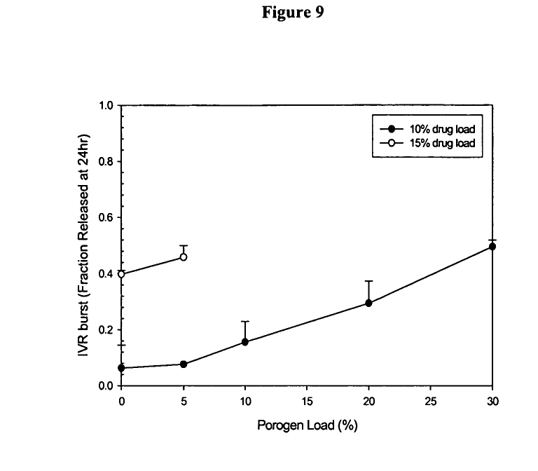

Microparticles with X% peptide A and Y% porogen were reconstituted in Dulbecco's phosphate buffer saline (PBS) and incubated at 37° C. under sink conditions. Half of the supernatant was subsequently removed and replenished with fresh PBS at each time point. The amount of drug released at each time-point was then quantified by RP-HPLC. The in vitro release (IVR) burst was determined as the cumulative fraction released at 24 hr. FIG. 9 shows cumulative fraction release of Peptide A at t=24 hr (IVR burst) as a function of porogen load for the 10% drug load and 15% drug load formulations; illustrating an increase in burst with porogen and drug load.

### Example 11

### Reduction of MP Burst with Higher Molecular Weight Polymers

Microparticles without porogen were fabricated as in Example 3 with the following difference: polymer molecular weight ranged from Mn of 1500 to 7900 Da. As in Example 10, the IVR burst is determined as the cumulative fraction released at 24 hr. Table 3 shows that the IVR burst of microparticles decreases with increased polymer molecular weight.

TABLE 3
Reduction of MP Burst with Higher Molecular Weight Polymer

| Polymer (# lots) | Mn (Da) | In vitro burst (%) |
|---|---|---|
| 5050 DL1A (n = 3) | 1500 | 57 ± 6 |
| 5050 DL2A (n = 2) | 4200 | 16 ± 3 |
| 5050 DL2.5A (n = 1) | 7900 | 1 |

We claim:

1. A composition comprising:
a) a biocompatible, biodegradable polymeric matrix;
b) between about 2% to about 20% (w/w) of a peptide dispersed and/or dissolved within the polymeric matrix; and
c) between about 5% to about 40% (w/w) of a carbohydrate component dispersed within the matrix, and
wherein the peptide is released from the matrix i) in a therapeutically effective amount for a defined release time period of about 3 days to about 21 days and ii) with a predetermined release pattern including an average initial burst release of less than 40% (w/w) of the peptide, when the composition is administered parenterally to a mammal.

2. The composition of claim 1 wherein the peptide is a B1 peptide antagonist.

3. The composition of claim 2 wherein the peptide is selected from SEQ ID NOS: 1-60 and an analog, conjugate, derivative, or pharmaceutically-acceptable salt form thereof.

4. The composition of claim 3 wherein the B1 peptide antagonist is selected from the peptides shown as SEQ ID NOS: 6-15, 33, 36, 37, and an analog, conjugate, derivative, or pharmaceutically-acceptable salt form thereof.

5. The composition of claim 2 wherein the peptide has the formula X-Arg Pro Hyp Gly Cpg Ser Dtic Cpg and X is selected from the group consisting of:
i) a D- or L-isomer of a natural or unnatural basic amino acid;
ii) a di- or tri- peptide of i); and
iii) an analog, conjugate, or derivative of i), or ii).

6. The composition of claim 5 wherein the carbohydrate component comprises at least 50% carbohydrate and about 0.1% to about 10% of at least one surfactant.

7. The composition of claim 6 wherein the carbohydrate component comprises at least 95% disaccharide.

8. The composition of claim 7 wherein the wherein the carbohydrate component comprises at least 95% trehalose.

9. The composition of claim 8 wherein the carbohydrate component comprises at least 99% trehalose.

10. The composition of claim 9 wherein the carbohydrate component comprises 1% sodium caprate.

11. The composition of claim 10 wherein the particles of the carbohydrate component have an average size between about 0.5 μm to about 5 μm.

12. The composition of claim 11 wherein the particles of the carbohydrate component have an average size between about 2 μm to about 5 μm.

13. The composition of claim 12 wherein the polymeric matrix comprises at least one polymer selected from poly(lactide), poly(glycolide), poly(lactide-co-glycolide), poly(lactic acid), poly(glycolic acid), poly(lactic acid-co-glycolic acid), polyanhydride, polyorthoester, polyetherester, polycaprolactone, polyesteramide, and copolymers and blends thereof.

14. The composition of claim 13 wherein the polymer comprises PLGA having a molecular weight from about 5 kD to about 20 kD.

15. The composition of claim 14 in a form selected from the group consisting of rods, pellets, cylinders, discs, and microparticles.

16. The composition of claim 15 wherein the form is microparticles.

17. The composition of claim 16 wherein effective amounts of the peptide is released for about 5 days to about 21 days.

18. The composition of claim 17 wherein effective amounts of the peptide is released for about 7 days to about 14 days.

19. The composition of claim 18 wherein effective amounts of the peptide is released for about 10 days.

20. The composition of claim 16 wherein the peptide is dispersed within the polymer.

21. A method of treating or preventing a B1 mediated disease, disorder, and/or condition comprising administering to a patient in need thereof a therapeutically effective amount of a sustained release composition comprising:
a) a biocompatible, biodegradable polymeric matrix;
b) between about 2% to about 20% (w/w) of a B1 peptide antagonist dispersed and/or dissolved within the polymeric matrix; and
c) between about 5% to about 40% (w/w) of a carbohydrate component dispersed within the matrix, and
wherein the peptide is released from the matrix i) in a therapeutically effective amount for a defined release time period of about 3 days to about 21 days and ii) with a predetermined release pattern including an initial average burst of less than 40%, when the composition is administered parenterally to a mammal.

22. The method of claim 21 wherein the peptide is selected from SEQ ID NOS: 1-60 and an analog, conjugate, derivative, or pharmaceutically-acceptable salt form thereof.

23. The method of claim 22 wherein the B1 peptide antagonist is selected from the peptides shown as SEQ ID NOS: 6-15, 33, 36, 37, and an analog, conjugate, derivative, or pharmaceutically-acceptable salt form thereof

24. The method of claim 21 wherein the peptide has the formula X-Arg Pro Hyp Gly Cpg Ser Dtic Cpg and X is selected from the group consisting of:
i) a D- or L-isomer of a natural or unnatural basic amino acid;
ii) a di- or tri- peptide of i); and
iii) an analog, conjugate, or derivative of i), or ii).

25. The method of claim 23 wherein the carbohydrate component comprises at least 50% carbohydrate and about 0.1% to about 10% of at least one surfactant.

26. The method of claim 25 wherein the carbohydrate component comprises at least 95% disaccharide.

27. The method of claim 26 wherein the carbohydrate component comprises at least 95% trehalose.

28. The method of claim 27 wherein the carbohydrate component comprises at least 99% trehalose.

29. The method of claim 27 wherein the carbohydrate component comprises at least 99% trehalose and 1% sodium caprate.

30. The method of claim 29 wherein the particles of the carbohydrate component have an average size between about 0.5 μm to about 5 μm.

31. The method of claim 30 wherein the particles of the carbohydrate component have an average size between about 2 μm to about 5 μm.

32. The method of claim 31 wherein the polymeric matrix comprises at least one polymer selected from poly(lactide), poly(glycolide), poly(lactide-co-glycolide), poly(lactic acid), poly(glycolic acid), poly(lactic acid-co-glycolic acid), polyanhydride, polyorthoester, polyetherester, polycaprolactone, polyesteramide, and copolymers and blends thereof.

33. The method of claim 32 wherein the polymer comprises PLGA having a molecular weight from about 5 kD to about 40 kD.

34. The method of claim 33 wherein the polymer comprises PLGA having a molecular weight from about 5 kD to about 20 kD.

35. The method of claim 34 in a form selected from the group consisting of rods, pellets, cylinders, discs, and microparticles.

36. The method of claim 35 wherein the form is microparticles.

37. The method of claim 36 wherein the microparticles are administered by injection.

38. The method of claim 37 wherein effective amounts of the peptide is released for about 5 days to about 21 days.

39. The method of claim 38 wherein effective amounts of the peptide is released for about 7 days to about 14 days.

40. The method of claim 39 wherein effective amounts of the peptide is released for about 10 days.

41. The method of claim 21 wherein the peptide is dispersed within the polymer.

42. The method of claim 39 wherein the B1 peptide antagonist is present from about 2% (w/w) to about 15% (w/w) of the total weight of the sustained release composition.

43. The method of claim 42 wherein the B1 peptide antagonist is present from about 5% (w/w) to about 10% (w/w) of the total weight of the sustained release composition.

44. The method of claim 43 wherein the B1 peptide antagonist is present at about 10% (w/w) of the total weight of the sustained release composition.

45. The method of claim 44, wherein the amount of carbohydrate in the carbohydrate component is about 5% (w/w) to about 20% (w/w) of the total dry weight of the sustained release composition.

46. The method of claim 45, wherein the carbohydrate is about 10% (w/w) of the total dry weight of the sustained release composition.

47. A method for preparing a composition for the sustained release of a B1 peptide antagonist comprising the steps of:
a) dissolving a poly(lactide-co-glycolide) copolymer having a molecular weight from about 5 kD to about 20 kD in a first solvent;
b) dissolving an amount of a peptide component comprising at least one B1 peptide antagonist in a second solvent such that the amount of the B1 peptide antagonists is between about 1% (w/w) and about 15% (w/w) of the dry weight of the composition;
c) mixing the polymer solution from a) and the peptide solution of b);
d) adding the mixture of c) to an amount of spray-dried particles of a carbohydrate component such that the amount of carbohydrate component is between about 5% to about 40% (w/w) of the dry weight of the composition;
e) forming microdroplets of the copolymer/peptide component/carbohydrate component mixture;
f) freezing the microdroplets;
g) extracting the solvents from the frozen microdroplets; and
h) filtering and drying the frozen droplets to obtain the microparticle composition.

48. A method for preparing a composition for the sustained release of a B1 peptide antagonist comprising the steps of:
a) dissolving a poly(lactide-co-glycolide) copolymer having a molecular weight from about 5 kD to about 20 kD in a first solvent;
b) dissolving an amount of a peptide component comprising at least one B1 peptide antagonist in a second solvent such that the total weight of the B1 peptide antagonists will be between about 1% (w/w) and about 15% (w/w) of the dry weight of the composition;
c) mixing the polymer solution from a) and the peptide solution of b);
d) adding the mixture of c) to an amount of spray-dried particles of a carbohydrate component such that the amount of carbohydrate component is between about 5% to about 40% (w/w) of the dry weight of the composition;
e) forming microdroplets of the copolymer/peptide component/carbohydrate component mixture;
f) spray dry the droplets; and
g) extracting the solvents from spray dried droplets; and
h) collecting the dried microparticles.

49. The method of claim 47 or 48 wherein the first solvent is selected from the group consisting of dimethysulfoxide, ethyl acetate, methylacetate, methylene chloride, chloroform, hexafluoroisopropanol, acetone, and combinations thereof and the second solvent is selected from the group consisting of ethanol, methanol, acetonitrile, DMF, DMSO, DCM, and combinations thereof.

50. The method of claim 49 wherein the first solvent is methylene chloride and the second solvent is methanol.

51. The method of claim 50 wherein the percentage of methanol in the mixture of c) is about 2% to about 20%.

52. The method of claim 51 wherein the percentage of methanol in the methanol:methylene chloride solution is between about 2% to about 10%.

53. The method of claim 52 wherein the percentage of methanol in the mixture of c) is between about 2% to about 8%.

54. The method of claim 53 wherein the percentage of methanol in the mixture of c) is between about 3% to about 6%.

55. The method of claim 54 wherein the percentage of methanol in the methanol:methylene chloride solution is from about 3% to about 4%.

56. The method of claim 55 wherein the carbohydrate component comprises between about 90% to about 99% trehalose.

57. The method of claim 56 wherein the carbohydrate component comprises between about 95% to about 99% trehalose.

58. The method of claim 57 wherein the carbohydrate component comprises about 99% trehalose. and about 1% sodium caprate.

59. A pharmaceutical composition comprising a composition according to claims 1-20 and a pharmaceutically-acceptable diluent or carrier.

60. The method of claim 21 wherein the B1 mediated disease, disorder, and/or condition is selected from the group consisting of pain, acute pain, dental pain, pain from trauma, surgical pain, pain from amputation or abscess, cancer, chronic alcoholism, stroke, thalamic pain syndrome, diabetes, acquired immune deficiency syndrome (“AIDS”), toxins and chemotherapy, general headache, migraine, cluster headache, mixed-vascular and non-vascular syndromes, tension headache, general inflammation, arthritis, rheumatic diseases, lupus, osteoarthritis, inflammatory bowel disorders, inflammatory eye disorders, inflammatory or unstable bladder disorders, psoriasis, skin complaints with inflammatory components, sunburn, carditis, dermatitis, myositis, neuritis, collagen vascular diseases, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, post-herpetic neuralgia, disturbances of visceral motility at respiratory, genitourinary, gastrointestinal or vascular regions, wounds, burns, allergic skin reactions, pruritis, vitiligo, general gastrointestinal disorders, colitis, gastric ulceration, duodenal ulcers, vasomotor or allergic rhinitis, and bronchial disorders

61. The method of claim 60 wherein pain arises from a disease disorder and/or condition selected from the group consisting of arthritis, rheumatoid arthritis, osteoarthritis, surgery, post-herpetic neuralgia, and diabetic neuropathy.

62. The composition produced according to the method of claim 47.


SEQUENCE LISTING (60 sequences)

SEQ ID NO: 1 - protein, 9 aa, Human
KRPPGFSPL

SEQ ID NO: 2 - protein, 10 aa, ARTIFICIAL SEQUENCE
  PEPTIDE
KKRPXGXSXX

SEQ ID NO: 3 - protein, 9 aa, ARTIFICIAL SEQUENCE
  PEPTIDE
XRPXGXSXX

SEQ ID NO: 4 - protein, 9 aa, ARTIFICIAL SEQUENCE
  PEPTIDE
XRPXGXSXX

SEQ ID NO: 5 - protein, 10 aa, ARTIFICIAL SEQUENCE
  PEPTIDE
XRPXGXSXXR

SEQ ID NO: 6 - protein, 10 aa, ARTIFICIAL SEQUENCE
  PEPTIDE
XRPXGXSXXR

SEQ ID NO: 7 - protein, 10 aa, ARTIFICIAL SEQUENCE
  PEPTIDE
XRPXGXSXXR

SEQ ID NO: 8 - protein, 9 aa, ARTIFICIAL SEQUENCE
  PEPTIDE
XRPXGXSXX

SEQ ID NO: 9 - protein, 10 aa, ARTIFICIAL SEQUENCE
  PEPTIDE
XRPXGXSXXR

SEQ ID NO: 10 - protein, 10 aa, ARTIFICIAL SEQUENCE
  PEPTIDE
KKRPPGXSXI

SEQ ID NO: 11 - protein, 10 aa, ARTIFICIAL SEQUENCE
  PEPTIDE
XRPXGXSXXR

SEQ ID NO: 12 - protein, 10 aa, ARTIFICIAL SEQUENCE
  PEPTIDE
KKRPXGXSXX

SEQ ID NO: 13 - protein, 10 aa, ARTIFICIAL SEQUENCE
  PEPTIDE
KKRPXGXSXX

SEQ ID NO: 14 - protein, 10 aa, ARTIFICIAL SEQUENCE
  PEPTIDE
XRPXGXSXXR

SEQ ID NO: 15 - protein, 10 aa, ARTIFICIAL SEQUENCE
  PEPTIDE
XKRPXGXSXX

SEQ ID NO: 16 - protein, 10 aa, ARTIFICIAL SEQUENCE
  PEPTIDE
XKRPXGXSXX

SEQ ID NO: 17 - protein, 10 aa, ARTIFICIAL SEQUENCE
  PEPTIDE
XKRPXGXSXX

SEQ ID NO: 18 - protein, 10 aa, ARTIFICIAL SEQUENCE
  PEPTIDE
XKRPXGXSXX

SEQ ID NO: 19 - protein, 9 aa, ARTIFICIAL SEQUENCE
  PEPTIDE
XRPXGXSXX

SEQ ID NO: 20 - protein, 9 aa, ARTIFICIAL SEQUENCE
  PEPTIDE
XRPXGXSXX

SEQ ID NO: 21 - protein, 9 aa, ARTIFICIAL SEQUENCE
  PEPTIDE
KRPXGXSXX

SEQ ID NO: 22 - protein, 10 aa, ARTIFICIAL SEQUENCE
  PEPTIDE
XKRPXGXSXX

SEQ ID NO: 23 - protein, 10 aa, ARTIFICIAL SEQUENCE
  PEPTIDE
KXRPXGXSXX

SEQ ID NO: 24 - protein, 10 aa, ARTIFICIAL SEQUENCE
  PEPTIDE
KXRPXGXSXX

SEQ ID NO: 25 - protein, 10 aa, ARTIFICIAL SEQUENCE
  PEPTIDE
KXRPXGXSXX

SEQ ID NO: 26 - protein, 10 aa, ARTIFICIAL SEQUENCE
  PEPTIDE
KXRPXGXSXX

SEQ ID NO: 27 - protein, 13 aa, ARTIFICIAL SEQUENCE
  PEPTIDE
CGGGKRPPGFSPL

SEQ ID NO: 28 - protein, 15 aa, ARTIFICIAL SEQUENCE
  PEPTIDE
CGGGGGKRPPGFSPL

SEQ ID NO: 29 - protein, 15 aa, ARTIFICIAL SEQUENCE
  PEPTIDE
CGGGGGKKRPGFSPL

SEQ ID NO: 30 - protein, 17 aa, ARTIFICIAL SEQUENCE
  PEPTIDE
CGGGGGKRKRPPGFSPL

SEQ ID NO: 31 - protein, 11 aa, ARTIFICIAL SEQUENCE
  PEPTIDE
CGKRPPGFSPL

SEQ ID NO: 32 - protein, 16 aa, ARTIFICIAL SEQUENCE
  PEPTIDE
CGGGGGKKRPPGXSXI

SEQ ID NO: 33 - protein, 16 aa, ARTIFICIAL SEQUENCE
  PEPTIDE
CGGGGGKKRPXGXSXX

SEQ ID NO: 34 - protein, 18 aa, ARTIFICIAL SEQUENCE
  PEPTIDE
CGGGGGGGKKRPXGXSXX

SEQ ID NO: 35 - protein, 16 aa, ARTIFICIAL SEQUENCE
  PEPTIDE
CGGGGGKKRPXGXSXX

SEQ ID NO: 36 - protein, 10 aa, ARTIFICIAL SEQUENCE
  PEPTIDE
KKRPXGXSXX

SEQ ID NO: 37 - protein, 10 aa, ARTIFICIAL SEQUENCE
  PEPTIDE
KKRPXGXSXX

SEQ ID NO: 38 - protein, 10 aa, ARTIFICIAL SEQUENCE
  PEPTIDE
CKRPPGFSPL

SEQ ID NO: 39 - protein, 16 aa, ARTIFICIAL SEQUENCE
  PEPTIDE
CGGGGGXKRPXGXSXX

SEQ ID NO: 40 - protein, 16 aa, ARTIFICIAL SEQUENCE
  PEPTIDE
CGGGGGXKRPXGXSXX

SEQ ID NO: 41 - protein, 16 aa, ARTIFICIAL SEQUENCE
  PEPTIDE
CGGGGGKXRPXGXSXX

SEQ ID NO: 42 - protein, 15 aa, ARTIFICIAL SEQUENCE
  PEPTIDE
GGGGGKKRPXGXSXX

SEQ ID NO: 43 - protein, 10 aa, ARTIFICIAL SEQUENCE
  PEPTIDE
XKRPXGXSXX

SEQ ID NO: 44 - protein, 10 aa, ARTIFICIAL SEQUENCE
  PEPTIDE
XKRPXGXSXX

SEQ ID NO: 45 - protein, 10 aa, ARTIFICIAL SEQUENCE
  PEPTIDE
XKRPXGXSXX

SEQ ID NO: 46 - protein, 10 aa, ARTIFICIAL SEQUENCE
  PEPTIDE
XKRPXGXSXX

SEQ ID NO: 47 - protein, 10 aa, ARTIFICIAL SEQUENCE
  PEPTIDE
XKRPXGXSXX

SEQ ID NO: 48 - protein, 10 aa, ARTIFICIAL SEQUENCE
  PEPTIDE
XKRPXGXSXX

SEQ ID NO: 49 - protein, 10 aa, ARTIFICIAL SEQUENCE
  PEPTIDE
XXRPXGXSXX

SEQ ID NO: 50 - protein, 10 aa, ARTIFICIAL SEQUENCE
  PEPTIDE
KXRPXGXSXX

SEQ ID NO: 51 - protein, 9 aa, ARTIFICIAL SEQUENCE
  PEPTIDE
XRXPGXSXI

SEQ ID NO: 52 - protein, 9 aa, ARTIFICIAL SEQUENCE
  PEPTIDE
XRXPGXSXI

SEQ ID NO: 53 - protein, 10 aa, ARTIFICIAL SEQUENCE
  PEPTIDE
XKRXPGXSXI

SEQ ID NO: 54 - protein, 10 aa, ARTIFICIAL SEQUENCE
  PEPTIDE
XKRXPGXSXI

SEQ ID NO: 55 - protein, 9 aa, ARTIFICIAL SEQUENCE
  PEPTIDE
KRPPGFSXI

SEQ ID NO: 56 - protein, 9 aa, ARTIFICIAL SEQUENCE
  PEPTIDE
KRPPGFSXI

SEQ ID NO: 57 - protein, 9 aa, ARTIFICIAL SEQUENCE
  PEPTIDE
XRXPGXSXI

SEQ ID NO: 58 - protein, 9 aa, ARTIFICIAL SEQUENCE
  PEPTIDE
XRXPGXSXI

SEQ ID NO: 59 - protein, 9 aa, ARTIFICIAL SEQUENCE
  PEPTIDE
KRXPGXSXI

SEQ ID NO: 60 - protein, 9 aa, ARTIFICIAL SEQUENCE
  PEPTIDE
KRXPGXSXI